(19) United States
(12) Patent Application Publication
(10) Pub. No.: US 2022/0193037 A1
(43) Pub. Date: Jun. 23, 2022

(54) PHARMACEUTICAL COMPOSITION FOR TOPICAL USE COMPRISING AT LEAST ONE AZOLATED LOCAL ANTIFUNGAL SUBSTANCE

(71) Applicant: SOCIETE D'EXPLOITATION DE PRODUITS POUR LES INDUSTRIES CHIMIQUES SEPPIC,, Paris Cedex 07 (FR)

(72) Inventors: Catherine BULCOURT, Paris Cedex 07 (FR); Alice DENIS, Paris Cedex 07 (FR); Séverine SIGURANI, Paris Cedex 07 (FR)

(21) Appl. No.: 17/601,325

(22) PCT Filed: Apr. 1, 2020
(86) PCT No.: PCT/EP2020/059300

§ 371 Date: Oct. 4, 2021

(30) Foreign Application Priority Data
Apr. 5, 2019 (FR) FR1903677

(51) Int. Cl. A61K 31/4174; A61K 9/00; A61K 9/107; A61K 31/496; A61K 31/4178; A61K 47/10; A61K 47/34

(52) U.S. Cl. CPC A61K 31/4174; A61K 9/0014; A61K 9/107; A61K 47/34; A61K 31/4178; A61K 47/10; A61K 31/496

(57) ABSTRACT

Disclosed is a pharmaceutical composition for topical. use in the form of a water-in-oil emulsion including, for 100% of the weight thereof: from 0.3% to 5% by weight of at least one azolated local antifungal substance;—from 60% to 90% by weight of a gelled aqueous phase (A1);—from 9.7% to 35% by weight of a fatty phase (A2) including at least one oil and an emulsifying system including a combination of at least one emulsifying surfactant (S1) selected from the elements of the group consisting of alkylpolyglycoside compositions and alkylpolyglycoside and fatty alcohol compositions, and of at least one emulsifying surfactant (S2) selected from the elements of the group consisting of polyglycerol esters, alkoxylated polyglycerol esters, polyglycol polyhydroxystearates, polyglycerol polyhydroxystearates and alkoxylated polyglycerol polyhydroxystearates.

### BACKGROUND OF THE INVENTION

### Field of the Invention

The present invention relates to a pharmaceutical composition for topical use which is in the form of a water-in-oil emulsion comprising at least one azole antifungal substance and is intended to be used by topical administration to the skin, the mucous membranes and/or the scalp of the human or animal body.

### Description of the Related Art

Dermopharmaceutical and pharmaceutical compositions can be in the form of aqueous solutions, emulsions and powders. Emulsions are particularly preferred forms since they make it possible to convey both the water-soluble substances and liposoluble substances that are frequently used in these applications. A distinction is made between oil-in-water (O/W) emulsions in which the continuous phase consists of a hydrophilic phase, generally an aqueous phase, and the dispersed phase consists of a lipophilic fatty phase, and water-in-oil (W/O) emulsions in which the continuous phase consists of a lipophilic fatty phase and the dispersed phase consists of a hydrophilic phase, generally an aqueous phase.

Oil-in-water emulsions are intrinsically more stable than water-in-oil emulsions; water-in-oil emulsions nevertheless have a number of advantages. Specifically, the separation between the water droplets reduces the possibility of the proliferation of microorganisms. Furthermore, the use of preserving agents, which is essential when the continuous phase is aqueous, can be avoided or reduced when the continuous phase is fatty. Water-in-oil emulsions are far less sensitive to low temperature than oil-in-water emulsions. Lastly, for topical applications for cosmetic use, the European patent application published under the number EP 1961455 A1 discloses that an oily continuous phase makes it possible to cover the skin after application of the water-in-oil emulsion, which protects the skin from dehydration and against external substances by forming a persistent oily film, thus making it possible to treat dry skin.

The solutions proposed in the prior art for preparing dermatological emulsions which are in the water-in-oil form are not satisfactory because either the silicone derivatives employed are volatile and may have harmful effects with regard to the environment and the users, or the silicone derivatives employed are not very volatile and then they impart unpleasant sensorial properties after topical application, such as for example sticky sensations on the skin. In addition, when they include specific therapeutic substances, for instance local antifungal substances such as local azole antifungal substances, these water-in-oil type emulsions show problems of stability during storage or even the impossibility of achieving an emulsion in stable water-in-oil form.

Proceeding from this, one problem is to develop a new composition for topical use which is in the form of a water-in-oil emulsion, which does not have the drawbacks exposed above, which remains homogeneous at ambient temperature (greater than or equal to 20° C. and less than or equal to 25° C.) and at 45° C. after storage for a minimum of three months, and which comprises a local antifungal substance.

### SUMMARY OF THE INVENTION

A solution of the present invention is a pharmaceutical composition (E1) for topical use which is in the form of a water-in-oil emulsion comprising:
from 0.3% to 5% by weight of at least one local azole antifungal substance;
from 60% to 90% by weight of a gelled aqueous phase (A1)
from 9.7% to 35% by weight of a fatty phase (A2) comprising at least one oil and an emulsifying system comprising a combination of at least one emulsifying surfactant (S1) chosen from the elements of the group consisting of compositions of alkyl polyglycosides, and compositions of alkyl polyglycosides and of fatty alcohols, and of at least one emulsifying surfactant (S2) chosen from the elements of the group consisting of polyglycerol esters, alkoxylated polyglycerol esters, polyglycol polyhydroxystearates, polyglycerol polyhydroxystearates, and alkoxylated polyglycerol polyhydroxystearates.

For the purposes of the present invention, the term “antifungal substance” means a substance or a chemical composition which makes it possible, once administered to a human or animal subject, to treat mycoses, that is to say infections caused by the action of microscopic fungi localized on a specific part of the body of a human or animal mammal.

An antifungal substance works either by directly attacking the fungal wall, thus causing the death of the cell (fungicidal action), or by blocking cell division, thus stopping the reproduction of fungi (fungistatic action).

For the purposes of the present invention, the term “local antifungal substance” denotes an “antifungal substance” as defined above and intended to treat superficial mycoses that are not very widespread and not far below the surface, and that are localized at the level of the skin of all parts of the body, scalp and mucous membranes.

For the purposes of the present invention, the term “mucous membrane” denotes the parts of the body of the human or animal mammal consisting of an epithelium and of underlying connective tissue (the chorion), separated by a basal lamina.

Among the antifungal substances, a distinction can be made between five different classes of substances: polyenes, echinocandins, azole compounds, fluoropyrimidines, allylamines

For the purposes of the present invention, the term “local azole antifungal substance” denotes a “local antifungal substance” as defined above which comprises in its molecular structure either an imidazolyl group of formula (A) or a triazolyl group of formula (B):

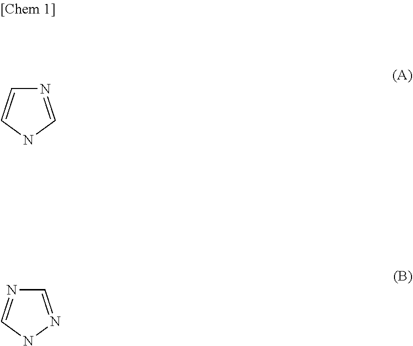 [SMILES: c1c[nH]cn1 ; c1nc[nH]n1]

said imidazolyl groups of formula (A) or triazolyl groups of formula (B) being linked to an asymmetric carbon.

The expression “for topical use” used in the definition of the pharmaceutical composition (El) according to the invention, which is in the form of a water-in-oil type emulsion as defined above, means that said pharmaceutical composition (El) is used by application to the skin, the hair, the scalp or the mucous membranes, whether it is a direct application in the case of a dermopharmaceutical or pharmaceutical composition or an indirect application for example in the case of a personal hygiene product in the form of a textile or paper wipe or of sanitary products intended to be in contact with the skin or the mucous membranes.

As appropriate, the pharmaceutical composition (El) for topical use according to the invention may comprise one or more of the following features:
the gelled aqueous phase comprises, for 100% of the weight thereof, from 0.5% to 10% by weight of a crosslinked anionic polyelectrolyte (AP), from 90% to 99.5% by weight of water. It is noted that preferably said composition comprises from 61.5% to 90% by weight, and more particularly still from 63% by weight to 90% by weight, of the gelled aqueous phase comprising, for 100% of the weight thereof: from 0.5% to 8.5% by weight, and more particularly still from 0.5% to 7% by weight, of a crosslinked anionic polyelectrolyte (AP), and from 91.5% to 99.5% by weight, and more particularly still from 93% to 99.5% by weight, of water;
the crosslinked anionic polyelectrolyte (AP) comprises a proportion of greater than or equal to 25 mol % of monomer units derived from 2-methyl-24(1-oxo-2-propenyl)aminol-1-propanesulfonic acid in free acid or partially or totally salified form,
the local azole antifungal substance is chosen from the elements, in a neutral form or in the form of salts, of the group consisting of: (R,S)-1-1-[phenyl(4-phenylphenyl)methyl]-1H-imidazole or bifonazole, in the form of the R enantiomer of formula (Iai) (CAS number =91487-85-3) or of the S enantiomer of formula (Ia₂) (CAS number =91487-86-4):

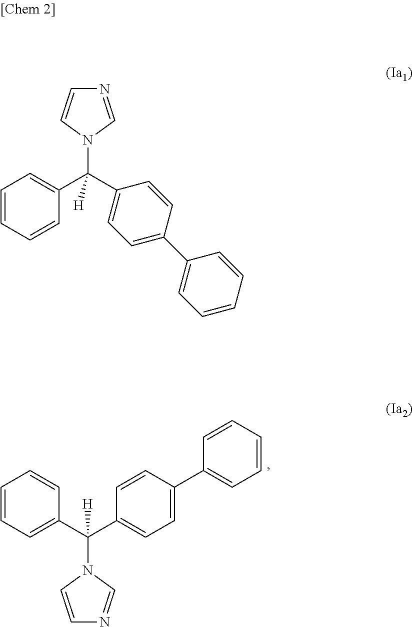 [SMILES: c1ccc(-c2ccc([C@@H](c3ccccc3)n3ccnc3)cc2)cc1 ; c1ccc(-c2ccc([C@H](c3ccccc3)n3ccnc3)cc2)cc1]

(R,S)-1-{2-[(4-chlorophenyl)methoxy]-2-(2,4-dichlorophenyl)ethyl}-1H-imidazole (CAS number=27220-47-9) or econazole, in the form of the R enantiomer of formula (Ib₁) or of the S enantiomer of formula (Ib2):

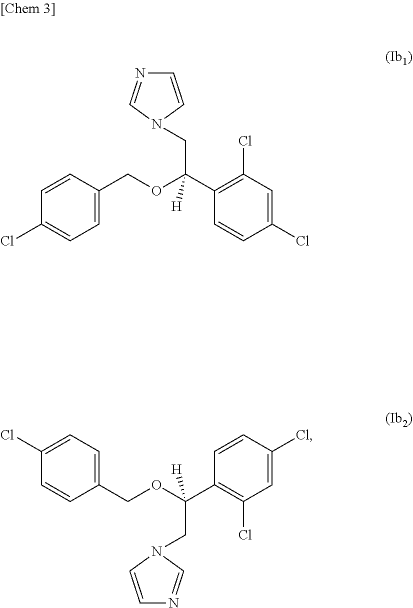 [SMILES: Clc1ccc(CO[C@@H](Cn2ccnc2)c2ccc(Cl)cc2Cl)cc1 ; Clc1ccc(CO[C@H](Cn2ccnc2)c2ccc(Cl)cc2Cl)cc1]

(R,S)-1-[2-(2,4-dichlorophenyl)-2-{[4-(phenylsulfanyl)phenyl]methoxy}ethyl]-1H-imidazole or fenticonazole (CAS number=72479-26-6) of formula (Ic):

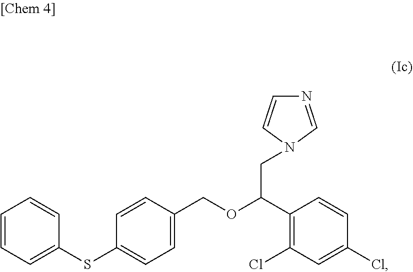 [SMILES: Clc1ccc(C(Cn2ccnc2)OCc2ccc(Sc3ccccc3)cc2)c(Cl)c1]

(R,S)-1-{2-[(2,6-dichlorobenzyl)oxy]-2-(2,4-dichlorophenyl)ethyl}-1H-imidazole or isoconazole (CAS number=27523-40-6) in the form of the R enantiomer of formula (Idi) or in the form of the S enantiomer of formula (Id₂):

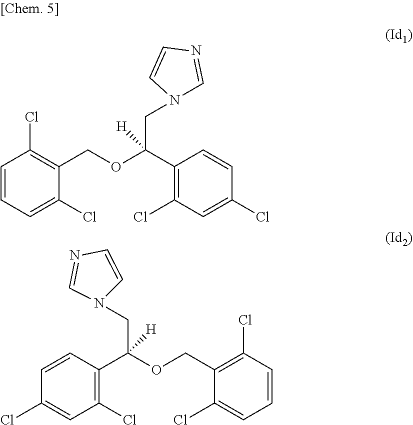

1-[4-(4-{[(2R,4S)-2-(2,4-dichlorophenyl)-2-(1H-imidazol-1-ylmethyl)-1,3-dioxolan-4-yl]methoxy}phenyl)piperazin-1-yl]ethan-1-one or ketoconazole (CAS number=65277-42-1) in the form of the R enantiomer of formula (Iei) or in the form of the S enantiomer of formula (Ie2):

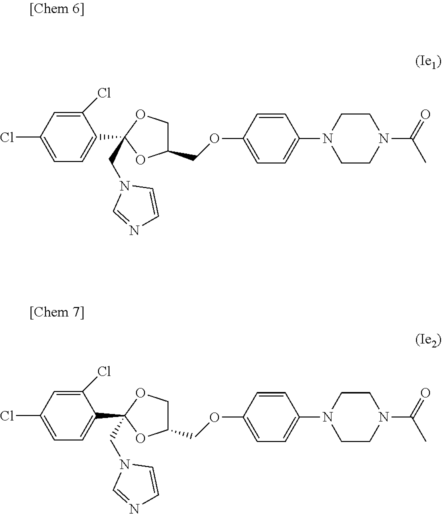 [SMILES: CC(=O)N1CCN(c2ccc(OC[C@@H]3CO[C@@](Cn4ccnc4)(c4ccc(Cl)cc4Cl)O3)cc2)CC1 ; CC(=O)N1CCN(c2ccc(OC[C@H]3CO[C@](Cn4ccnc4)(c4ccc(Cl)cc4Cl)O3)cc2)CC1]

(R,S)-1-{2-[(2,4-dichlorobenzyl)oxy]-2-(2,4-dichlorophenyl)ethyl}-1H-imidazole or miconazole (CAS number=22916-47-8) in the form of the R enantiomer of formula (If₁) or in the form of the S enantiomer of formula (If₂):

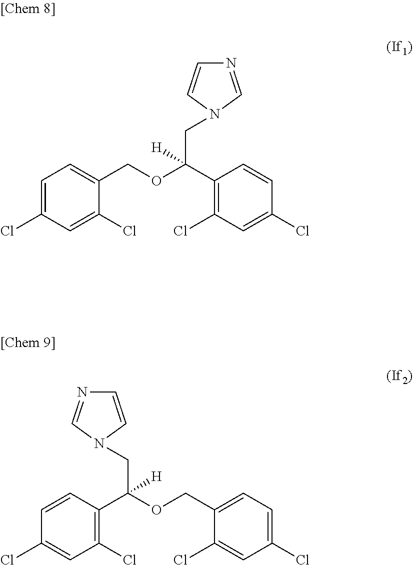 [SMILES: Clc1ccc(CO[C@@H](Cn2ccnc2)c2ccc(Cl)cc2Cl)c(Cl)c1 ; Clc1ccc(CO[C@H](Cn2ccnc2)c2ccc(Cl)cc2Cl)c(Cl)c1]

1-[(Z)-2-[2-(4-chlorophenoxy)ethoxy]-2-(2,4-dichlorophenyl)-1-methylvinyl]-1H-imidazole (CAS number=74512-12-2) or omoconazole of formula (Ig):

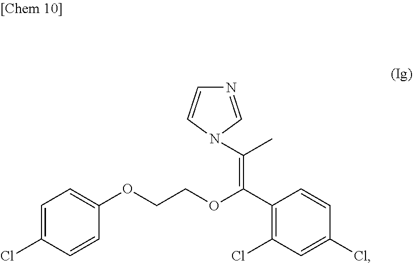 [SMILES: C/C(=C(/OCCOc1ccc(Cl)cc1)c1ccc(Cl)cc1Cl)n1ccnc1]

(1Z)-N-R2,4-dichlorobenzyl)oxyl-1-(2,4-dichlorophenyl)-2-(1H-imidazol-1-yl)ethanimine or oxiconazole (CAS number=64211-45-6) of formula (Ih):

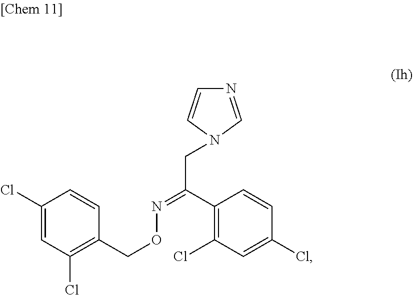 [SMILES: Clc1ccc(CO/N=C(/Cn2ccnc2)c2ccc(Cl)cc2Cl)c(Cl)c1]

(R,S)-1-{2-[(7-chloro-1-benzothiophen-3-yl)methoxy]-2-(2,4-dichlorophenyl)ethyl}-1H-imidazole or sertaconazole (CAS number=99592-32-2) of formula (Ii):

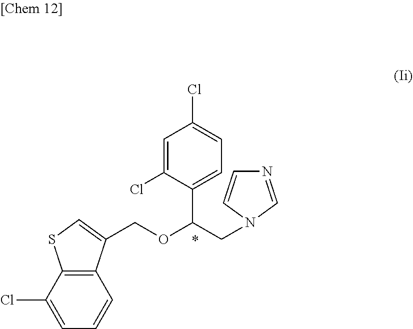 [SMILES: Clc1ccc(C(Cn2ccnc2)OCc2csc3c(Cl)cccc23)c(Cl)c1]

(R,S)-1-{2-[(2-chlorothiophen-3-yl)methoxy]-2-(2,4-dichlorophenyl)ethyl}-1H-imidazole or tioconazole (CAS number=65899-73-2) in the form of the R enantiomer of formula (Ij₁) or in the form of the S enantiomer of formula (Ij₂):

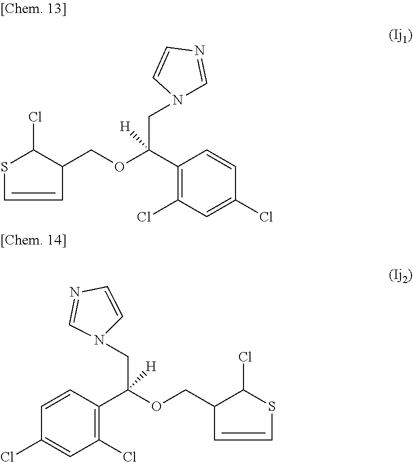 [SMILES: Clc1ccc([C@H](Cn2ccnc2)OCC2C=CSC2Cl)c(Cl)c1 ; Clc1ccc([C@@H](Cn2ccnc2)OCC2C=CSC2Cl)c(Cl)c1]

1-[(2-chlorophenyl)(diphenyl)methyl]-1H-imidazole or clotrimazole (CAS number=23593-75-1) of formula (1k)

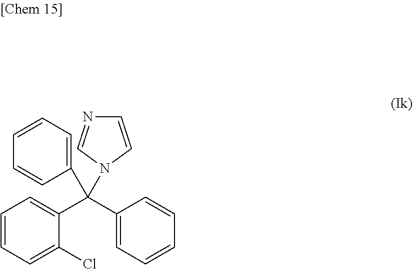 [SMILES: Clc1ccccc1C(c1ccccc1)(c1ccccc1)n1ccnc1]

the local azole antifungal substance is chosen from the elements of the group consisting of the hydrochloride, phosphate, nitrate, propionate and citrate salts of the compounds of formulae (Ia), (Ib), (Ic₁), (Ic₂), (Id), (Ie), (If), (Ig), (Ih), (Ii), (Ij₁), (Ij₂) and (Ik); preferably, the local azole antifungal substance is the hydrochloride or nitrate salt of an element of the group consisting of (R,S)-1-{2-[(4-chlorophenyl)methoxy]-2-(2,4-dichlorophenyl)ethyl}-1H-imidazole (CAS number=27220-47-9) or econazole, in the form of the R enantiomer of formula (Ib₁) or in the form of the S enantiomer of formula (Ib₂), 1-[4-(4-{[(2R,4S)-2-(2,4-dichlorophenyl)-2-(1H-imidazol-1-ylmethyl)-1,3-dioxolan-4-yl]methoxy}phenyl)piperazin-1-yl]ethan-1-one or ketoconazole (CAS number=65277-42-1) in the form of the R enantiomer of formula (Ie₁) or in the form of the S enantiomer of formula (Ie₂), and 1-[(2-chlorophenyl)(diphenyl)methyl]-1H-imidazole or clotrimazole (CAS number=23593-75-1) of formula (Ik).
the emulsifying surfactant (S1) consists of at least one alkyl polyglycoside composition (C1) represented by formula (VIII):

R1-O-(G)x-H (VIII)
wherein x represents a decimal number between 1.05 and 2.5, G represents the glucosyl or α,β-D-glucopyranosyl radical, obtained from the removal of the hemiacetal hydroxyl group from α,β-D-glucopyranose, and R1 represents a radical chosen from the elements of the group consisting of the radicals n-dodecyl, n-tetradecyl, n-hexadecyl, n-octadecyl, n-eicosyl and n-behenyl, said composition (C1) consisting of a mixture of compounds represented by formulae (VIII₁), (VIII₂), (VIII₃), (VIII₄) and (VIII₅):

R1-O-(G)₁-H (VIII₁)

R1-O-(G)₂-H (VIII₂)

R1-O-(G)₃-H (VIII₃)

R1-O-(G)₄-H (VIII₄)

R1-O-(G)₅-H (VIII₅)
in the respective molar proportions a₁, a₂, a₃, a₄ and as, such that:
the sum a₁+a₂+a₄+a₅ is equal to 1, and that
the sum a₁+2a₂+3a₃+4a₄+5a₅ is equal to x.
the emulsifying surfactant (S1) consists of at least one composition (C2) comprising, for 100% of the weight thereof:
   from 10% to 50% by weight of at least one alkylpolyglycoside composition (C1) represented by formula (VIII):

R1-O-(G)x-H (VIII)

wherein x represents a decimal number between 1.05 and 2.5, G represents the glucosyl or α,β-D-glucopyranosyl radical, obtained from the removal of the hemiacetal hydroxyl group from α,β-D-glucopyranose, and R1 represents a radical chosen from the elements of the group consisting of the radicals n-dodecyl, n-tetradecyl, n-hexadecyl, n-octadecyl, n-eicosyl and n-behenyl, said composition (C1) consisting of a mixture of compounds represented by formulae (VIII₁), (VIII₂), (VIII₃), (VIII₄) and (VIII₅):

R1-O-(G)₁-H (VIII₁)

R1-O-(G)₂-H (VIII₂)

R1-O-(G)₃-H (VIII₃)

R1-O-(G)₄-H (VIII₄)

R1-O-(G)₅-H (VIII₅)
in the respective molar proportions a₁, a₂, a₃, a₄ and a₅, such that:
the sum a₁+a₂+a₃+a₄+a₅ is equal to 1, and that
the sum a₁+2a₂+3a₃+4a₄+5a₅ is equal to x; and from 90% to 50% by weight of at least one fatty alcohol of formula (IX):

R″1—OH (IX),

wherein R″1 represents a radical chosen from the elements of the group consisting of the radicals n-dodecyl, n-tetradecyl, n-hexadecyl, n-octadecyl, n-eicosyl and n-behenyl, where R′1 may be identical to or different from R1;
the emulsifying surfactant (S2) consists of at least one polyglycol polyhydroxystearate represented by formula (XII):

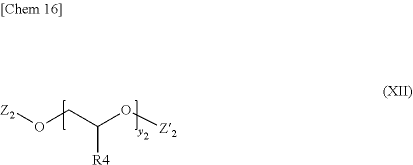

wherein y2 represents an integer greater than or equal to 2 and less than or equal to 50, R4 represents the hydrogen atom, the methyl radical or the ethyl radical, Z2 represents a radical of formula (XIII):

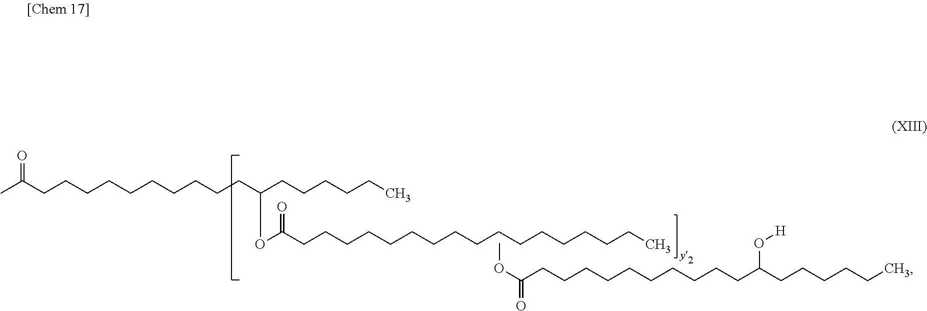

wherein y′2 represents an integer greater than or equal to 0 and less than or equal to 10, more particularly greater than or equal to 1 and less than or equal to 10, and Z2 represents a radical of formula (XIII) as defined above, with Z′2 being identical to or different from Z2, or a hydrogen atom;
the weight ratio between the emulsifying surfactant (S1) and the emulsifying surfactant (S2) is greater than or equal to ¼ and greater than or equal to 1, more preferentially greater than or equal to ⅓ and less than or equal to 1, more preferentially still greater than or equal to ⅓ and less than or equal to ½.
said composition is intended for use in therapy in human or animal mammals.
said composition is for use in treating mycoses of the skin, the scalp, the mouth and/or the gynecological system in human or animal mammals.

More particularly, said composition is for use in treating mycoses of the skin such as, for example, cutaneous candidiasis, dermatophytoses (Trichophyton, Epidermophyton, Microsporum), pityriasis versicolor, erythrasma, “athlete's foot” or toe-web intertrigo; vulvovaginal mycoses, whether or not superinfected by Gram+ bacteria; candida vaginitis.
said composition additionally comprises at least one or more auxiliary compounds chosen from foaming and/or detergent surfactants, thickening and/or gelling surfactants, thickening and/or gelling agents, stabilizers, film-forming compounds, solvents and cosolvents, hydrotropic agents, plasticizing agents, opacifying agents, pearlescent agents, superfatting agents, sequestering agents, chelating agents, antioxidants, fragrances, essential oils, preservatives, conditioning agents, deodorants, mineral fillers or pigments.

The composition according to the invention can generally comprise excipients and/or active principles commonly used in the field of formulations for topical, in particular pharmaceutical or dermopharmaceutical, use.

Preferably, the fatty phase (A2) comprises, for 100% of the weight thereof:
from 1.25% to 25% by weight, more particularly from 1.25% to 20% by weight, of an emulsifying system (S) comprising, for 100% by weight of said emulsifying system (S):
from 12% to 88% by weight, more particularly from 15% to 85% by weight, and more particularly still from 20% by weight to 85% by weight, of at least one emulsifying surfactant (Si), and
from 12% to 88% by weight, more particularly from 15% to 85% by weight, and more particularly still from 20% by weight to 85% by weight, of at least one emulsifying surfactant (S2);
from 75% to 98.75% by weight, more particularly from 80% to 98.75% by weight, and more particularly still from 82% to 98.75% by weight, of at least one oil and optionally of at least one wax.
   For the purposes of the present invention, the expression “salts of the elements composed of the group consisting of the compounds of formulae (Ia₁), (Ia₂), (Ib₁), (Ib₂), (Ic), (Idi), (Id2), (Ie₁), (Ie₂), (If₂), (Ig), (Ih), (Ii), (Ij 1), (Ij₂) and (Ik)” means the substances obtained by adding acids acceptable for pharmaceutical use to said compounds of formulae (Ia₁), (Ia₂), (Ib₁), (Ib₂), (Ic), (Id₁), (Id₂), (Ie₁), (Ie₂), (If₂), (Ig), (Ih), (Ii), (Ij₂) and (Ik).

As acids acceptable for pharmaceutical use, mention may be made of hydrohalic acids, such as for example hydrochloric acid and hydrobromic acid and more particularly hydrochloric acid; sulfuric acid, phosphoric acid, nitric acid, perchloric acid; aliphatic, alicyclic, aromatic or heterocyclic sulfonic or carboxylic acids, such as for example formic acid, acetic acid, propionic acid, succinic acid, glycolic acid, lactic acid, malonic acid, tartaric acid, citric acid, ascorbic acid, maleic acid, hydroxymaleic acid, pyruvic acid, phenylacetic acid, benzoic acid, p-aminobenzoic acid, anthranilic acid, p-hydroxybenzoic acid, salicylic acid or p-aminosalicylic acid, methanesulfonic and ethanesulfonic acid, hydroxyethanesulfonic acid, ethylenesulfonic acid, halobenzenesulfonic acids, toluenesulfonic acid, naphthalene-1,5-disulfonic acid or sulfanilic acid, methionine, tryptophan, lysine or arginine.

According to one particular aspect, these acids which are acceptable for pharmaceutical use that can be added to the compounds of formulae (Ia₁), (Ia₂), (Ib₁), (Ib₂), (Ic), (Id₁), (Id₂), (Ie₁), (Ie₂), (If₂), (Ig), (Ih), (Ii), (Ij₁), (Ij₂) and (Ik) so as to obtain therefrom the resulting salts are chosen from the elements of the group consisting of hydrochloric acid, phosphoric acid, nitric acid, propionic acid, citric acid.

The addition of hydrochloric acid to the compounds of formulae (Ia₁), (Ia₂), (Ib₁), (Ib₂), (Ic), (Id₁), (Id₂), (Ie₁), (Ie₂), (If₁), (If₂), (Ig), (Ih), (Ii), (h₁), (Ij₂) and (Ik) makes it possible to obtain salts known as hydrochlorides of said compounds.

The addition of phosphoric acid to the compounds of formulae (Ia₁), (Ia₂), (Ib₁), (Ib₂), (Ic), (Id₁), (Id₂), (Ie₁), (Ie₂), (If₂), (Ig), (Ih), (Ii), (Ij₁), (Ij₂) and (Ik) makes it possible to obtain salts known as phosphates of said compounds.

The addition of nitric acid to the compounds of formulae (Ia₁), (Ia₂), (Ib₁), (Ib₂), (Ic), (Id₁), (Id₂), (Ie₁), (Ie₂), (If₁), (If₂), (Ig), (Ih), (Ii), (Ij₁), (Ij₂) and (Ik) makes it possible to obtain salts known as nitrates of said compounds.

The addition of propionic acid to the compounds of formulae (Ia₁), (Ia₂), (Ib₁), (Ib₂), (Ic), (Id₂), (Ie₁), (Ie₂), (If₁), (If₂), (Ig), (Ih), (Ii), (Ij₁), (Ij2) and (Ik) makes it possible to obtain salts known as propionates of said compounds.

The addition of citric acid to the compounds of formulae (Ia₁), (Ia₂), (Ib₁), (Ib₂), (Ic), (Id₁), (Id₂), (Ie₁), (Ie₂), (If₁), (If₂), (Ig), (Ih), (Ii), (Ij₁), (Ij₂) and (Ik) makes it possible to obtain salts known as citrates of said compounds.

The term “oil” denotes a compound and/or a mixture of compounds that are water-insoluble, and liquid at 25° C., and more particularly:
linear alkanes including from 11 to 19 carbon atoms;
branched alkanes comprising from 7 to 40 carbon atoms, such as isododecane, isopentadecane, isohexadecane, isoheptadecane, isooctadecane, isononadecane or isoeicosane, or mixtures of some of them, such as those mentioned below and identified by their INCI name: C₇₋₈ isoparaffin, C₈₋₉ isoparaffin, C₉₋₁₁ isoparaffin, C₉₋₁₂ isoparaffin, C₉₋₁₃ isoparaffin, C₉₋₁₄ isoparaffin, C₉₋₁₆ isoparaffin, C₁₀₋₁₂ isoparaffin, C₁₀₋₁₂ isoparaffin, C₁₀₋₁₃ isoparaffin, C₁₁₋₁₂ isoparaffin, C₁₁₋₁₃ isoparaffin, C₁₁₋₁₄ isoparaffin, C₁₂₋₁₄ isoparaffin, C₁₂₋₂₀ isoparaffin, C₁₃₋₁₄ isoparaffin, C₁₃₋₁₆ isoparaffin;
cycloalkanes optionally substituted with one or more linear or branched alkyl radicals;
white mineral oils, such as the products sold under the following names: Marcol™ 52, Marcol™ 82, Drakeol™ 6VR, Eolane™ 130, Eolane™ 150;
hemisqualane (or 2,6,10-trimethyldodecane; CAS number: 3891-98-3), squalane (or 2,6,10,15,19,23-hexamethyltetracosane), hydrogenated polyisobutene or hydrogenated polydecene;
mixtures of alkanes including from 15 to 19 carbon atoms, said alkanes being linear alkanes, branched alkanes and cycloalkanes, and more particularly the mixture (M₁) which comprises, for 100% of the weight thereof, a proportion by weight of branched alkanes of greater than or equal to 90% and less than or equal to 100%; a proportion by weight of linear alkanes of greater than or equal to 0% and less than or equal to 9%, and more particularly less than 5%, and a proportion by weight of cycloalkanes of greater than or equal to 0% and less than or equal to 1%, for example the mixtures sold under the name Emogreen™ L15 or Emogreen™ L19;
the fatty alcohol ethers of formula (II):

Z₁—O—Z₂ (II),
wherein Z₁ and Z₂, which may be identical or different, represent a linear or branched alkyl radical including from 5 to 18 carbon atoms, for example dioctyl ether, didecyl ether, didodecyl ether, dodecyl octyl ether, dihexadecyl ether, (1,3-dimethylbutyl) tetradecyl ether, (1,3-dimethylbutyl) hexadecyl ether, bis(1,3-dimethylbutyl) ether or dihexyl ether.
the monoesters of fatty acids and of alcohols of formula (III):

R′₁—(C═O)—O—R₂ (III),

wherein R′₁—(C═O) represents a saturated or unsaturated, linear or branched acyl radical comprising from 8 to 24 carbon atoms, and R′2 represents, independently of R′₁, a saturated or unsaturated, linear or branched hydrocarbon-based chain comprising from 1 to 24 carbon atoms, for example methyl laurate, ethyl laurate, propyl laurate, isopropyl laurate, butyl laurate, 2-butyl laurate, hexyl laurate, methyl cocoate, ethyl cocoate, propyl cocoate, isopropyl cocoate, butyl cocoate, 2-butyl cocoate, hexyl cocoate, methyl myristate, ethyl myristate, propyl myristate, isopropyl myristate, butyl myristate, 2-butyl myristate, hexyl myristate, octyl myristate, methyl palmitate, ethyl palmitate, propyl palmitate, isopropyl palmitate, butyl palmitate, 2-butyl palmitate, hexyl palmitate, octyl palmitate, methyl oleate, ethyl oleate, propyl oleate, isopropyl oleate, butyl oleate, 2-butyl oleate, hexyl oleate, octyl oleate, methyl stearate, ethyl stearate, propyl stearate, isopropyl stearate, butyl stearate, 2-butyl stearate, hexyl stearate, octyl stearate, methyl isostearate, ethyl isostearate, propyl isostearate, isopropyl isostearate, butyl isostearate, 2-butyl isostearate, hexyl isostearate, isostearyl isostearate;
the diesters of fatty acids and of glycerol of formula (IV) and of formula (V):

R′₃—(C═O)—O—CH₂—CH(OH)—CH₂—O—(C═O)—R′₄ (IV)

R′₅—(C═O)—O—CH₂—CH[O—(C═O)—R′₆]—CH₂—OH (V),

formulae (IV) and (V) wherein R′₃—(C═O), R′₄—(C═O), R′₅—(C═O) and R′₆—(C═O), which may be identical or different, represent a saturated or unsaturated, linear or branched acyl group including from 8 to 24 carbon atoms;
triesters of fatty acids and of glycerol of formula (VI):

R′₇—(C═O)—O—CH₂—CH[O—(C═O)—R″₈]—CH₂—O—(C═O)—R″₉ (VI),

wherein R′₇—(C═O), R′₈—(C═O) and R′₉—(C═O), which may be identical or different, represent a saturated or unsaturated, linear or branched acyl group including from 8 to 24 carbon atoms.
plant oils, such as phytosqualane, sweet almond oil, coconut kernel oil, castor oil, jojoba oil, olive oil, rapeseed oil, groundnut oil, sunflower oil, wheat germ oil, corn germ oil, soybean oil, cottonseed oil, alfalfa oil, poppy seed oil, pumpkin seed oil, evening primrose oil, millet oil, barley oil, rye oil, safflower oil, candlenut oil, passionflower oil, hazelnut oil, palm oil, shea butter, apricot kernel oil, beauty-leaf oil, sisymbrium oil, avocado oil, calendula oil, and oils derived from flowers or vegetables;
ethoxylated plant oils.

Preferably, the composition according to the invention comprises at least one oil chosen from the elements of the group consisting of castor oil, liquid paraffins, cocoyl caprylate/caprate, isopropyl myristate and capric/caprylic triglyceride.

The fatty phase (A2) optionally comprises wax. The latter is more particularly chosen from beeswax, carnauba wax, candelilla wax, ouricury wax, Japan wax, cork fiber wax, sugarcane wax, paraffin waxes, lignite waxes, microcrystalline waxes, lanolin wax; ozokerite; polyethylene wax; silicone waxes; vegetable waxes; fatty alcohols and fatty acids that are solid at ambient temperature; glycerides that are solid at ambient temperature.

In the definition of the composition (E1) for topical use which is a subject of the present invention, the term “crosslinked anionic polyelectrolyte (AP)” denotes a non-linear crosslinked anionic polyelectrolyte, which is in the form of a three-dimensional network which is insoluble in water but swellable in water and which leads to the production of a chemical gel.

Preferably, the crosslinked anionic polyelectrolyte (AP) comprises, for 100 mol %:

(a1)—a proportion of greater than or equal to 25 mol % and less than or equal to 100 mol % of monomer units derived from 2-methyl-2-[(1-oxo-2-propenyl)amino]-1-propanesulfonic acid in free acid or partially or totally salified form;
(a2)—optionally, a proportion of greater than 0 mol % and less than or equal to 75 mol % of monomer units derived from at least one monomer chosen from the elements of the group consisting of acrylamide, N,N-dimethylacrylamide; methacrylamide or N-isopropylacrylamide;
(a3)—optionally, a proportion of greater than 0 mol % and less than or equal to 20 mol %, more particularly greater than 0 mol % and less than or equal to 15 mol %, more particularly still greater than or equal to 0 mol % and less than or equal to 10 mol % of monomer units derived from at least one monomer chosen from the elements of the group consisting of (2-hydroxyethyl) acrylate, (2,3-dihydroxypropyl) acrylate, (2-hydroxyethyl) methacrylate, (2,3-dihydroxypropyl) methacrylate and vinylpyrrolidone
(a4)—optionally, a proportion of greater than 0 mol % and less than or equal to 75 mol % of monomer units derived from at least one monomer chosen from the elements of the group consisting of acrylic acid, methacrylic acid, 2-carboxyethylacrylic acid, itaconic acid, maleic acid and 3-methyl-3-[(1-oxo-2-propenyl)amino]mbutanoic acid, the carboxylic function of said monomers being in free acid, partially salified or totally salified form;
(a5)—optionally, a proportion of greater than 0 mol % and less than or equal to 5 mol % of at least one monomer of formula (VII):

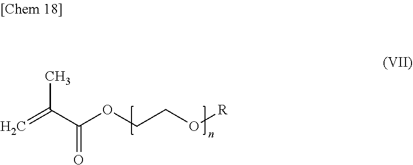

wherein R represents a linear or branched alkyl radical including from 8 to 20 carbon atoms and n represents an integer greater than or equal to 0 and less than or equal to 20;
(a6)—a proportion of greater than 0 mol % and less than or equal to 1 mol % of monomer units derived from at least one diethylenic or polyethylenic cros slinking monomer (AR); the sum of said molar proportions of the monomer units according to a1), a2), a3), a4), a5) and a6) being equal to 100 mol %.

For the purposes of the present invention, the term “salified” indicates that the acid function present in a monomer is in an anionic form associated in salt form with a cation, in particular alkali metal salts, such as sodium or potassium cations, or such as nitrogenous base cations, such as the ammonium salt, the lysine salt or the monoethanolamine salt (HOCH₂—CH₂—NH₄⁺). They are preferably sodium or ammonium salts.

The term “at least one diethylenic or polyethylenic crosslinking monomer (AR)” notably denotes, in the definition of said crosslinked anionic polyelectrolyte (AP), a monomer chosen from the elements of the group consisting of methylenebis(acrylamide), ethylene glycol dimethacrylate, diethylene glycol diacrylate, ethylene glycol diacrylate, diallylurea, triallylamine, trimethylolpropane triacrylate, diallyloxyacetic acid or a salt thereof, such as sodium diallyloxyacetate, or a mixture of these compounds; and more particularly a monomer chosen from ethylene glycol dimethacrylate, triallylamine, trimethylolpropane triacrylate or methylenebis(acrylamide) or a mixture of these compounds.

According to another particular aspect of the present invention, the composition (E₁) for topical use is characterized in that said crosslinking monomer (AR) as defined previously is used in a molar proportion of less than or equal to 0.5%, more particularly less than or equal to 0.25% and most particularly less than or equal to 0.1%; it is more particularly greater than or equal to 0.005 mol %.

The crosslinked anionic polyelectrolyte (AP) used in the composition (E₁) for topical use which is a subject of the present invention may also comprise various additives such as complexing agents, transfer agents or chain-limiting agents.

The crosslinked anionic polyelectrolyte (AP) used in the composition (E₁) for topical use which is a subject of the present invention may be prepared by performing a radical polymerization process known to those skilled in the art, for instance processes of solution polymerization, suspension polymerization, inverse suspension polymerization, emulsion polymerization, inverse emulsion polymerization or polymerization in solvent medium followed by a step of precipitation of the polymer formed.

According to a more particular aspect, the crosslinked anionic polyelectrolyte (AP) used in the composition (E₁) for topical use which is a subject of the present invention may be prepared by performing a process of polymerization in solvent medium followed by a step of precipitation of the polymer formed, or of inverse emulsion polymerization optionally followed by a step of concentration and/or atomization.

According to a more particular aspect, the crosslinked anionic polyelectrolyte (AP) used in the composition (E₁) for topical use which is a subject of the present invention may be prepared according to one of the processes described above and may involve the use of transfer agents or chain-limiting agents. The transfer agents or chain-limiting agents are more particularly chosen from the group consisting of sodium hypophosphite, alcohols of low molecular weight, for example methanol, ethanol, 1-propanol, isopropanol or butanol, thiols, for example 2-mercaptoethanol, transfer agents comprising a sulfate function, for example sodium methallylsulfonate, or mixtures of said transfer agents. The transfer agents or chain-limiting agents are more particularly used in molar proportions, expressed relative to the total number of moles of monomers used, of from 0.001 mol % to 1 mol %, more particularly from 0.001 mol % to 0.5 mol %, and most particularly from 0.001 mol % to 0.1 mol %.

According to another particular aspect of the present invention, said crosslinked anionic polyelectrolyte (AP) is an element of the group consisting of a homopolymer of 2-methyl-2-[(1-oxo-2-propenyl)-amino]-1-propanesulfonic acid partially or totally salified in sodium salt or ammonium salt form, crosslinked with triallylamine and/or methylenebis(acrylamide); a copolymer of 2-methyl-2-[(1-oxo-2-propenyl)amino]-1-propanesulfonic acid partially or totally salified in sodium salt or ammonium salt form and of acrylic acid partially or totally salified in sodium salt or ammonium salt form, crosslinked with triallylamine and/or methylenebis(acrylamide); a copolymer of 2-methyl-2-[(1-oxo-2-propenyl)amino]-1-propanesulfonic acid (γ) partially or totally salified in sodium salt or ammonium salt form and of acrylic acid (δ) partially or totally salified in sodium salt form in a molar ratio (γ)/(δ) of greater than or equal to 30/70 and less than or equal to 90/10, crosslinked with triallylamine and/or methylenebis(acrylamide); a copolymer of 2-methyl-2-[(1-oxo-2-propenyl)amino]-1-propanesulfonic acid (γ) partially or totally salified in sodium salt form, and of acrylic acid (δ) partially or totally salified in sodium salt form in a molar ratio (γ)/(δ) of greater than or equal to 40/60 and less than or equal to 90/10, crosslinked with triallylamine and/or methylenebis(acrylamide); a copolymer of 2-methyl-2-[(1-oxo-2-propenyl)amino]-1-propanesulfonic acid (γ) partially or totally salified in sodium salt form, and of acrylamide (ε) in a molar ratio (γ)/(ε) of greater than or equal to 30/70 and less than or equal to 90/10, crosslinked with triallylamine and/or methylenebis(acrylamide); a copolymer of 2-methyl-2-[(1-oxo-2-propenyl)amino]-1-propanesulfonic acid (γ) partially or totally salified in sodium salt form, and of hydroxyethyl acrylate (ζ) in a molar ratio (γ)/(ζ) of greater than or equal to 30/70 and less than or equal to 90/10, crosslinked with triallylamine and/or methylenebis(acrylamide);

a terpolymer crosslinked with triallylamine and/or methylenebis(acrylamide), of 2-methyl-2-[(1-oxo-2-propenyl)amino]-1-propanesulfonic acid partially or totally salified in sodium salt or ammonium salt form, of acrylamide and of acrylic acid partially or totally salified in sodium salt or ammonium salt form; a terpolymer crosslinked with triallylamine and/or methylenebis(acrylamide), of 2-methyl-2-[(1-oxo-2-propenyl)amino]-1-propanesulfonic acid partially or totally salified in sodium salt or ammonium salt form in a molar proportion of greater than or equal to 30% and less than or equal to 45%, of acrylamide in a molar proportion of greater than or equal to 45% and less than or equal to 68% and of acrylic acid partially or totally salified in sodium salt or ammonium salt form in a molar proportion of greater than or equal to 2% and less than or equal to 10%; a terpolymer crosslinked with triallylamine and/or methylenebis(acrylamide), of 2-methyl-2-[(1-oxo-2-propenyl)amino]-1-propanesulfonic acid partially or totally salified in sodium salt or ammonium salt form in a molar proportion of greater than or equal to 30% and less than or equal to 45%, of acrylamide in a molar proportion of greater than or equal to 47% and less than or equal to 68% and of acrylic acid partially or totally salified in sodium salt or ammonium salt form in a molar proportion of greater than or equal to 2% and less than or equal to 8%; a terpolymer crosslinked with trimethylolpropane triacrylate and/or triallylamine and/or methylenebis(acrylamide), of 2-methyl-2-[(1-oxo-2-propenyl)amino]-1-propanesulfonic acid partially or totally salified in sodium salt or ammonium salt form in a molar proportion of greater than or equal to 60% and less than or equal to 80%, of N,N-dimethylacrylamide in a molar proportion of greater than or equal to 15% and less than or equal to 39.5% and of tetraethoxylated lauryl methacrylate in a molar proportion of greater than or equal to 0.5% and less than or equal to 5%; a tetrapolymer crosslinked with trimethylolpropane triacrylate and/or triallylamine and/or methylenebis(acrylamide), of 2-methyl-2-[(1-oxo-2-propenyl)amino]-1-propanesulfonic acid partially or totally salified in sodium salt or ammonium salt form in a molar proportion of greater than or equal to 60% and less than or equal to 80%, of N,N-dimethylacrylamide in a molar proportion of greater than or equal to 15% and less than or equal to 39%, of lauroyl methacrylate in a molar proportion of greater than or equal to 0.5% and less than or equal to 2.5%, and of stearoyl methacrylate in a molar proportion of greater than or equal to 0.5% and less than or equal to 2.5%.

Regarding the emulsifying surfactant, in formula (VIII) as defined above, the group R1—O— is linked to G via the anomeric carbon of the saccharide residue, so as to form an acetal function.

According to one particular aspect, in the definition of formula (VIII) x, or mean degree of polymerization, represents a decimal number greater than or equal to 1.05 and less than or equal to 2.5, more particularly greater than or equal to 1.05 and less than or equal to 2.0 and more particularly still greater than or equal to 1.25 and less than or equal to 2.0.

According to another particular aspect, the combination of at least one emulsifying surfactant (S1) and at least one emulsifying surfactant (S2) comprises, for 100% of the weight thereof:
from 12% to 88% by weight of at least one emulsifying surfactant (S₁) which consists of at least one composition (C₂) comprising, for 100% of the weight thereof:
from 10% to 50% by weight of at least one alkyl polyglycoside composition (C₁represented by formula (VIII):

R₁-O-(G)ₓ-H (VIII)
wherein x represents a decimal number between 1.05 and 2.5, G represents the glucosyl or α,β-D-glucopyranosyl radical, obtained from the removal of the hemiacetal hydroxyl group from α,β-D-glucopyranose, and R₁ represents a radical chosen from the elements of the group consisting of the radicals n-dodecyl, n-tetradecyl, n-hexadecyl, n-octadecyl, n-eicosyl and n-behenyl, said composition consisting of a mixture of compounds represented by formulae (VIII₁), (VIII₂), (VIII₃), (VIII₄) and (VIII₅):

R₁-O-(G)₁-H (VIII₁)

R₁-O-(G)₂-H (VIII₂)

R₁-O-(G)₃-H (VIII₃)

R₁-O-(G)₄-H (VIII₄)

R₁-O-(G)₅-H (VIII₅)

in the respective molar proportions a₁, a₂, a₃, a₄ and a₅, such that:
the sum a₁+a₂+a₃+a₄+a₅ is equal to 1, and that
the sum a₁+2a₂+3a₃+4a₄+5a₅ is equal to x; and
from 90% to 50% by weight of at least one fatty alcohol of formula (IX):

R″₁—OH (IX),
wherein R″₁ represents a radical chosen from the elements of the group consisting of the radicals n-dodecyl, n-tetradecyl, n-hexadecyl, n-octadecyl, n-eicosyl and n-behenyl, where R′₁ may be identical to or different from R₁
from 12% to 88% by weight of at least one emulsifying surfactant (S₂) which consists of at least one polyglycol polyhydroxystearate represented by formula (XII):

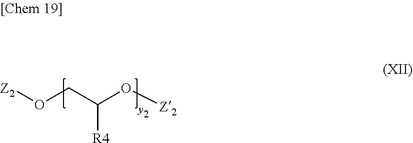

wherein y₂ represents an integer greater than or equal to 2 and less than or equal to 50, R₄ represents a hydrogen atom, a methyl radical or an ethyl radical, and Z₂ represents a radical of formula (XIII):

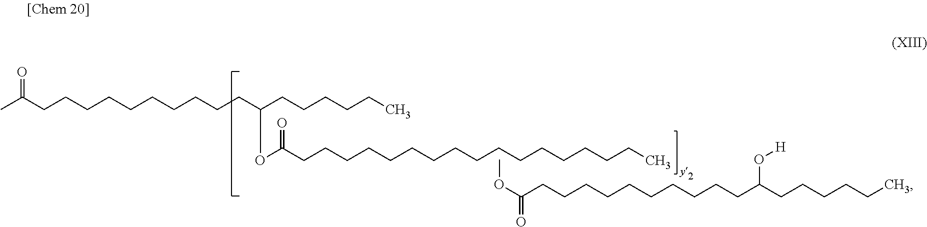

wherein y′₂ represents an integer greater than or equal to 0 and less than or equal to 10, more particularly greater than or equal to 1 and less than or equal to 10, and Z′₂ represents a radical of formula (XIII) as defined above, where Z₂′ may be identical to or different from Z₂, or a hydrogen atom.
According to another particular aspect, the composition according to the invention has a dynamic viscosity, measured at a temperature of 20° C. using a Brookfield LVT viscometer at a speed of 6 rpm, greater than or equal to 500 mPa·s and less than or equal to 40 000 mPa·s.
Regarding the auxiliary compounds, among the foaming and/or detergent anionic surfactants that may be combined with the composition according to the invention, mention may be made of alkali metal salts, alkaline-earth metal salts, ammonium salts, amine salts or amino alcohol salts of alkyl ether sulfates, of alkyl sulfates, of alkylamido ether sulfates, of alkylaryl polyether sulfates, of monoglyceride sulfates, of alpha-olefin sulfonates, of paraffin sulfonates, of alkyl phosphates, of alkyl ether phosphates, of alkyl sulfonates, of alkylamide sulfonates, of alkylaryl sulfonates, of alkyl carboxylates, of alkyl sulfosuccinates, of alkyl ether sulfosuccinates, of alkylamide sulfosuccinates, of alkyl sulfoacetates, of alkyl sarcosinates, of acyl isethionates, of N-acyl taurates, of acyl lactylates, of N-acylated derivatives of amino acids, of N-acylated derivatives of peptides, of N-acylated derivatives of proteins, or of fatty acids. Among the foaming and/or detergent amphoteric surfactants optionally present in the composition according to the invention, mention may be made of alkylbetaines, alkylamidobetaines, sultaines, alkylamidoalkylsulfobetaines, imidazoline derivatives, phosphobetaines, amphopolyacetates and amphopropionates.

Among the foaming and/or detergent cationic surfactants optionally present in the composition according to the invention, mention may be made particularly of quaternary ammonium derivatives.

Among the foaming and/or detergent nonionic surfactants optionally present in the composition according to the invention, mention may be made more particularly of alkyl polyglycosides containing a linear or branched, saturated or unsaturated aliphatic radical and comprising from 8 to 12 carbon atoms; castor oil derivatives, polysorbates, coconut kernel amides and N-alkylamines.

As examples of thickening and/or gelling surfactants optionally present in the composition according to the invention, mention may be made of:
optionally alkoxylated fatty esters of alkylpolyglycosides, and most particularly ethoxylated esters of methylpolyglucoside such as PEG 120 methyl glucose trioleate and PEG 120 methyl glucose dioleate sold, respectively, under the names Glucamate™ LT and Glumate™ DOE120;
alkoxylated fatty esters such as the PEG 150 pentaerythrityl tetrastearate sold under the name Crothix·8 DS53, or PEG 55 propylene glycol oleate sold under the name Antil™ 141;
fatty-chain polyalkylene glycol carbamates such as PPG 14 laureth isophoryl dicarbamate sold under the name Elfacos™ T211, or PPG 14 palmeth 60 hexyl dicarbamate sold under the name Elfacos™ GT2125.

As examples of emulsifying surfactants optionally present in the composition according to the invention, mention may be made of nonionic surfactants, anionic surfactants and cationic surfactants.

As examples of emulsifying nonionic surfactants optionally present in the composition according to the invention, ethoxylated castor oil and ethoxylated hydrogenated castor oil, for example the product sold under the name Simulsol™ 989; compositions comprising glycerol stearate and stearic acid poly(ethoxylated) with between 5 mol and 150 mol of ethylene oxide, for example the composition comprising stearic acid (ethoxylated) with 135 mol of ethylene oxide and glycerol stearate sold under the name Simulsol™ 165; ethoxylated sorbitan esters, for example the products sold under the name Montanox™; ethoxylated mannitan esters; sucrose esters; methyl glucoside esters.

As examples of emulsifying anionic surfactants optionally present in the composition according to the invention, mention may be made of decyl phosphate, cetyl phosphate sold under the name Amphisol™, glyceryl stearate citrate; cetearyl sulfate; the arachidyl/behenyl phosphates and arachidyl/behenyl alcohols composition sold under the name Sensanov™ WR; soaps, for example sodium stearate or triethanolammonium stearate, or N-acylated derivatives of amino acids which are salified, such as for example stearoyl glutamate.

As examples of emulsifying cationic surfactants optionally present in the composition according to the invention, mention may be made of amine oxides, quaternium-82, cetyltrimethylammonium chloride, hexadecyltrimethylammonium bromide, cetylpyridinium chloride, benzalkonium chloride, benzethonium chloride, and the surfactants described in document WO96/00719 and mainly those wherein the fatty chain comprises at least 16 carbon atoms.

As examples of opacifying agents and/or pearlescent agents optionally present in the composition according to the invention, mention may be made of sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate and fatty alcohols comprising from 12 to 22 carbon atoms.

As examples of texturing agents optionally present in the composition according to the invention, mention may be made of N-acylated derivatives of amino acids, for example lauroyl lysine sold under the name Aminohope™ LL, octenyl starch succinate sold under the name Dryflo™, myristyl polyglucoside sold under the name Montanov 14, cellulose fibers, cotton fibers, chitosan fibers, talc, sericite and mica.

As examples of solvents and co-solvents optionally present in the composition according to the invention, mention may be made of water, organic solvents, for example glycerol, diglycerol, glycerol oligomers, ethylene glycol, propylene glycol, butylene glycol, hexylene glycol, diethylene glycol, xylitol, erythritol, sorbitol, water-soluble alcohols such as ethanol, isopropanol or butanol, mixtures of water and of said organic solvents, propylene carbonate, ethyl acetate, benzyl alcohol and dimethyl sulfoxide (DMSO).

As examples of agents for improving the skin penetration optionally present in the composition according to the invention, mention may be made of glycol ethers, for instance ethylene glycol monomethyl ether, ethylene glycol monoethyl ether, ethylene glycol monopropyl ether, ethylene glycol monoisopropyl ether, ethylene glycol monobutyl ether, ethylene glycol monophenyl ether, ethylene glycol monobenzyl ether, diethylene glycol monomethyl ether, diethylene glycol monoethyl ether and diethylene glycol mono-n-butyl ether, diethylene glycol monoethyl ether (or Transcutol-P), fatty acids such as oleic acid, fatty acid esters of glycerol, for instance glyceryl behenate, glyceryl palmitostearate, behenoyl macroglycerides, polyoxyethylene-2-stearyl ether, polyoxyethylene-2-oleyl ether, terpenes, for instance D-limonene, and essential oils, for instance the essential oil of eucalyptus.

As examples of thickening and/or gelling agents optionally present in the composition according to the invention, mention may be made of polysaccharides consisting only of monosaccharides, such as glucans or glucose homopolymers, glucomannoglucans, xyloglycans, galactomannans of which the degree of substitution (DS) of the D-galactose units on the main D-mannose chain is between 0 and 1, and more particularly between 1 and 0.25, such as galactomannans originating from cassia gum (DS=⅕), locust bean gum (DS=¼), tara gum (DS=⅓), guar gum (DS=½) or fenugreek gum (DS=1).

As examples of thickening and/or gelling agents optionally present in the composition according to the invention, mention may be made of polysaccharides consisting of monosaccharide derivatives, such as sulfated galactans and more particularly carrageenans and agar, uronans and more particularly algins, alginates and pectins, heteropolymers of monosaccharides and uronic acids, and more particularly xanthan gum, gellan gum, gum arabic exudates and karaya gum exudates, and glucosaminoglycans.

As examples of thickening and/or gelling agents optionally present in the composition according to the invention, mention may be made of cellulose, cellulose derivatives such as methyl cellulose, ethyl cellulose, hydroxypropyl cellulose, silicates, starch, hydrophilic starch derivatives, and polyurethanes.

As examples of stabilizers optionally present in the composition according to the invention, mention may be made of microcrystalline waxes, and more particularly of ozokerite, and mineral salts such as sodium chloride or magnesium chloride.

As examples of thermal or mineral waters which can be combined with the composition according to the invention, mention may be made of thermal or mineral waters having a mineralization of at least 300 mg/I, in particular Avene water, Vittel water, Vichy basin water, Uriage water, La Roche-Posay water, La Bourboule water, Enghien-les-Bains water, Saint-Gervais-les-Bains water, Néris-les-Bains water, Allevard-les-Bains water, Digne water, Maizières water, Neyrac-les-Bains water, Lons-le-Saunier water, Rochefort water, Saint Christau water, Les Fumades water and Tercis-les-Bains water.

As examples of active agents which can be combined with the composition according to the invention, mention may be made of substances or compositions which provide a beneficial effect to the human or animal subject.

These active agents may for example be antibodies, analgesics, anti-inflammatories, cytokines, cytoxins, growth factors, hormones, lipids, oligonucleotides, polymers, polysaccharides, polypeptides, protease inhibitors, vitamins, insect repellents, antibiotics or anti-inflammatory agents.

As examples of analgesic and anti-inflammatory agents which can be combined with the composition according to the invention, mention may be made of acetaminophen, aspirin, salicylic acid, methyl salicylate, choline salicylate, glycol salicylate, 1-menthol, camphor, mefenamic acid, fluphenamic acid, indomethacin, protizidic acid, fentiazac, tolmetin, tiaprofenic acid, phenylbutazone, oxyphenbutazone, clofezone, pentazocin, mepirizole, hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone and betamethasone.

As examples of nonsteroidal anti-inflammatory agents (or NSAIDs) which can be combined with the composition according to the invention, mention may more particularly be made of arylacetic (or arylalkanoic) derivatives and 2-arylpropionic acids (or profens), and even more particularly diclofenac, tiaprofenic acid, alminoprofen, etodolac, flurbiprofen, ibuprofen, ketoprofen, naproxen.

As examples of antiseptic agents which can be combined with the composition according to the invention, mention may be made of cetrimide, povidone-iodine, chlorhexidine, iodine, benzalkonium chloride, benzoic acid, nitrofurazone, benzoyl peroxide, hydrogen peroxide, hexachlorophene, phenol, resorcinol and cetylpyridinium chloride.

As examples of insecticidal agents which can be combined with the composition according to the invention, mention may be made of trichlorfon, triflumuron, fenthion, bendiocarb, cyromazine, diflubenzuron, dicyclanil, fluazuron, amitraz, deltamethrin, cypermethrin, chlorfenvinphos, flumethrin, ivermectin, abamectin, avermectin, doramectin, moxidectin, zeta-cypermethrin, diazinon, spinosad, imidacloprid, nitenpyram, pyriproxyfen, fipronil, cythioate, lufenuron, selamectin, milbemycin oxime, chlorpyrifos, coumaphos, propetamphos, alpha-cypermethrin, cypermethrin high cis, ivermectin, diflubenzuron, cyclodiene, carbamate and benzoylurea.

As examples of antimicrobial agents which can be combined with the composition according to the invention, mention may be made of sulfonamides, aminoglycosides such as for example neomycin, tobramycin, gentamicin, amikacin, kanamycin, spectinomycin, paromomycin, netilmicin, polypeptides, cephalosporins, oxazolidinones such as for example ciprofloxacin, levofloxacin, and ofloxacin.

As examples of active agents which can be combined with the composition according to the invention, mention may be made of vitamin E, coenzyme Q10, L-carnitine, choline, folic acid, magnesium and its salts, caprylic acid, linoleic acid, lauric acid, taurine, vitamin C, vitamin A and vitamins of group B.

The composition according to the invention and as defined above is obtained by performing the preparation process comprising the following steps:

A step a) of preparing the fatty phase (A2) by mixing all of the elements constituting it in the desired proportions. This mixing step is generally performed at a temperature of greater than or equal to 20° C. and less than or equal to 80° C., more particularly greater than or equal to 25° C. and less than or equal to 80° C., and even more particularly greater than or equal to 30° C. and less than or equal to 80° C.; it is performed with mechanical stirring at a moderate speed of greater than or equal to 50 rpm and less than or equal to 100 rpm;

A step b) of preparing the aqueous phase (A₁) from all of the elements constituting it in the desired proportions. This mixing step is generally performed at a temperature of greater than or equal to 20° C. and less than or equal to 80° C., more particularly greater than or equal to 20° C. and less than or equal to 60° C., and even more particularly greater than or equal to 20° C. and less than or equal to 40° C.; it is performed with mechanical stirring at a moderate speed of greater than or equal to 500 rpm and less than or equal to 3000 rpm. In particular, the aqueous phase (A₁) obtained on conclusion of step b) has a dynamic viscosity, measured at 20° C. using a Brookfield LV viscometer at a speed of 6 rpm, of greater than or equal to 200 mPa·s and less than or equal to 40 000 mPa·s, more particularly greater than or equal to 1000 mPa·s and less than or equal to 40 000 mPa·s, and even more particularly greater than or equal to 2000 mPa·s and less than or equal to 40 000 mPa·s;

A step c) during which the fatty phase (A2) is added to the aqueous phase (A₁) at a temperature of greater than or equal to 20° C. and less than or equal to 80° C., more particularly greater than or equal to 20° C. and less than or equal to 60° C., and more particularly still greater than or equal to 20° C. and less than or equal to 40° C., with mechanical stirring at a moderate speed of greater than or equal to 50 rpm and less than or equal to 400 rpm, so as to obtain the composition according to the invention.
The examples that follow illustrate the invention without, however, limiting it.

### DESCRIPTION OF THE PREFERRED EMBODIMENTS

Preparation and evaluation of water-in-oil emulsions according to the invention and of comparative water-in-oil emulsions.

### 1) Preparation of the Water-In-Oil Emulsions

Three water-in-oil emulsions according to the invention, denoted (F₁) to (F₅), and three water-in-oil emulsions according to the prior art, denoted (F′₁) to (F₄), the proportions by weight of the constituents of which are recorded in table 1 below, the contents by weight of the polyelectrolytes being indicated as a percentage of polymeric solids, are prepared by performing the following process.

The constituents of the fatty phase are introduced successively into a beaker, mixed and brought to a temperature of 20° C. after an 80° C. heating step; the mixing is performed using a mechanical stirrer equipped with a propeller-type stirring spindle at a speed of 100 rpm. The glycerol and water are mixed at ambient temperature in a beaker using a mechanical stirrer at a speed of 2000 rpm and the thickener is then added gradually. The stirring is maintained for a duration which makes it possible to obtain an aqueous phase which is in the form of a homogeneous gel. The fatty phase is added in one go to the aqueous gel at ambient temperature and at a moderate stirring speed (75 to 300 rpm) using a stirrer equipped with an anchor-type spindle. This stirring is then maintained for 10 minutes and no cooling step is necessary.

TABLE 1

| | (F₁) | (F₂) | (F₃) | (F′₁) | (F′₂) | (F′₃) | (F₄) | (F₅) | (F′₄) |
|---|---|---|---|---|---|---|---|---|---|
| Fatty phase | | | | | | | | | |
| Econazole | 1% | 0% | 0% | 0% | 1% | 1% | 1% | 1% | 1% |
| nitrate | | | | | | | | | |
| Clotrimazole | 0% | 1% | 1% | 0% | 0% | 0% | 0% | 0% | 0% |
| Lanol ™ | 15% | 15% | 8% | 15% | 8% | 15% | 15% | 15% | 15% |
| 2681⁽¹⁾ | | | | | | | | | |
| Sepineo ™ SE | 1% | 3% | 3% | 1% | 1% | 0% | 0% | 0% | 0% |
| 68⁽²⁾ | | | | | | | | | |
| Montanov ™ | 0% | 0% | 0% | 0% | 0% | 0% | 1% | 0% | 0% |
| L⁽⁸⁾ | | | | | | | | | |
| Montanov ™ | 0% | 0% | 0% | 0% | 0% | 0% | 0% | 1% | 0% |
| 202⁽⁹⁾ | | | | | | | | | |
| Sepicide ™ | 1% | 1% | 1% | 1% | 1% | 1% | 1% | 1% | 1% |
| HB⁽³⁾ | | | | | | | | | |
| Simaline ™ | 3% | 3% | 3% | 3% | 3% | 3% | 3% | 3% | 3% |
| WO⁽⁴⁾ | | | | | | | | | |
| Montane ™ | 0% | 0% | 0% | 0% | 0% | 1.2% | 0% | 0% | 0% |
| 80⁽⁶⁾ | | | | | | | | | |
| Aqueous | | | | | | | | | |
| phase | | | | | | | | | |
| Sepineo ™ | 3% | 3% | 3% | 3% | 3% | 0% | 3% | 3% | |
| P600⁽⁵⁾ | | | | | | | | | |
| PEG-400⁽⁷⁾ | 25% | 15% | 15% | 25% | 25% | 25% | 25% | 25% | 3% |
| Ethanol | 8% | 8% | 8% | 8% | 8% | 8% | 8% | 8% | 25% |
| Water | q.s. | q.s. | q.s. | q.s. | q.s. | q.s. | q.s. | q.s. | 8% |
| | 100% | 100% | 100% | 100% | 100% | 100% | 100% | 100% | |

⁽¹⁾Land ™ 2681, or Coco-Caprylate/Caprate.
⁽²⁾Sepineo ™ SE 68 is a mixture comprising, for 100% of the weight thereof, from 78% to 85% by weight of a mixture of n-hexadecanol and n-octadecanol, and from 15% to 22% by weight of a mixture of n-hexadecyl glucoside with a mean degree of polymerization of 1.20 and n-octadecyl glucoside with a mean degree of polymerization of 1.20, used as emulsifying agent.
⁽³⁾Sepicide ™ HB is a mixture of phenoxyethanol, methylparaben, ethylparaben, butylparaben and n-propylparaben, used as a preservative.
⁽⁴⁾Simaline ™ WO, or PEG 30 Dipolyhydroxystearate, is an emulsifying surfactant.
⁽⁵⁾Sepineo ™ P600 is a self-invertible inverse latex comprising, for 100% of the weight thereof, between 30% and 40% by weight of a crosslinked copolymer of acrylamide and of sodium acryloyldimethyltaurate, used as a thickening agent.
⁽⁶⁾Montane ™ 80 is a composition comprising sorbitan monooleate, used as water-in-oil type emulsifying agent.
⁽⁷⁾PEG-400 is a polyethylene glycol with a molecular weight of approximately 400 g · mol⁻¹.

2 Demonstration of the properties of the water-in-oil emulsions (F₁) to (F₃) according to the invention and of the water-in-oil emulsions (F′₁) to (F′₃) according to the prior art. 2.1 Characterization of the appearance and the viscosity of the water-in-oil emulsions (F₁) to (F₃) according to the invention and of the water-in-oil emulsions (F′₁) to (F′₃) according to the prior art.

The emulsions (F₁) to (F₃) and (F′₁) to (F′₃) obtained according to the process described above are then stored in an insulated climatic chamber regulated at a temperature of 25° C. for 3 months. After the conclusion of this period of three months, the appearance (APP) of each emulsion prepared is observed and the dynamic viscosity (μ) of each emulsion is measured (in mPas) by means of a viscometer at 25° C. (Brookfield LVT, speed 6).

An aliquot of these same emulsions (F₁) to (F₃) and (F′₁) to (F′₃) obtained according to the process described above are also stored in an insulated climatic chamber regulated at a temperature of 45° C. for three months. After the conclusion of this period of three months, the appearance (APP) of each emulsion prepared is observed and the dynamic viscosity (μ) of each emulsion is measured (in mPas) by means of a viscometer at 25° C. (Brookfield LVT, speed 6).

2.2 Characterization of the direction of the emulsions (F₁) to (F₃) according to the invention and of the emulsions (F′₁) to (F′₃) according to the prior art.

The conductivity (σ) of the emulsions (F₁) to (F₃) according to the invention and of the emulsions (F′₁) to (F′₃) is measured at 25° C., after a period of storage of said emulsions of one day in an insulated climatic chamber regulated at a temperature of 25° C., by means of an LF 196™ brand conductivity meter from the company WTW equipped with a TetraCon™ 96 electrode. If, for a given emulsion, (σ)≤0.5 μS·cm⁻¹, the emulsion is considered to be non-conductive and consequently the external phase is considered not to be the aqueous phase but the oily phase.

If, for a given emulsion, (σ)>0.5 μS·cm⁻¹, the emulsion is considered to be conductive and consequently the external phase is considered not to be the oily phase but the aqueous phase.

This same measurement of the conductivity of the emulsions (F₁) to (F₃) according to the invention and of the emulsions (F′₁) to (F′₃) is measured at 25° C. after three months at 25° C., and after three months at 45° C.

2.3 Results obtained for the water-in-oil emulsions (F₁) to (F₃) according to the invention and for the water-in-oil emulsions (F′₁) to (F′₃) according to the prior art.

The evaluation methods described in paragraphs 2.1 and 2.2 were applied to the water-in-oil emulsions (F₁) to (F₃) according to the invention and to the water-in-oil emulsions (F′₁) to (F′₃) according to the prior art. The results obtained are recorded in table 2 below.

TABLE 2

| | (F₁) | (F₂) | (F3) | (F′₁) | (F′₂) |
|---|---|---|---|---|---|
| (APP) at 1 | Homogeneous | Homogeneous | Homogeneous | Homogeneous | Homogeneous |
| day | liquid | liquid | liquid | liquid | liquid |
| (visual) | | | | | O/W |
| (σ) at 1 | ≤0.5 μS · cm⁻¹ | ≤0.5 μS · cm⁻¹ | ≤0.5 μS · cm⁻¹ | ≤0.5 μS · cm⁻¹ | >0.5 μS · cm |
| day at | | | | | |
| 25° C. | | | | | |
| (APP) at 7 | Homogeneous | Homogeneous | Homogeneous | Homogeneous | Homogeneous |
| days at | liquid | liquid | liquid | liquid | liquid |
| 25° C. | | | | | O/W |
| (σ) at 3 | ≤0.5 μS · cm⁻¹ | ≤0.5 μS · cm⁻¹ | ≤0.5 μS · cm⁻¹ | ≤0.5 μS · cm⁻¹ | >0.5 μS · cm |
| months at | | | | | |
| 25° C. | | | | | |

| | (F′₃) | (F4) | (F5) | (F′₄) |
|---|---|---|---|---|
| (APP) at 1 | Homogeneous | Homogeneous | Homogeneous | Homogeneous |
| day | liquid | liquid | liquid | liquid |
| (visual) | O/W | | | |
| (σ) at 1 | >0.5 μS · cm | ≤0.5 μS · cm⁻¹ | ≤0.5 μS · cm⁻¹ | ≤0.5 μS · cm⁻¹ |
| day at | | | | |
| 25° C. | | | | |
| (APP) at 7 | Homogeneous | Homogeneous | Homogeneous | Liquid with |
| days at | liquid | liquid | liquid | slight |
| 25° C. | O/W | | | exudation |
| (σ) at 3 | >0.5 μS · cm⁻¹ | >0.5 μS · cm⁻¹ | >0.5 μS · cm⁻¹ | Not measured |
| months at | | | | |
| 25° C. | | | | |

2.4 Analysis of the results

The water-in-oil emulsions (F₁) to (F₃) according to the invention are therefore characterized by:
a stability of their water-in-oil form after 3 months of storage at a temperature of 25° C.; the observed appearance after this period of storage is still homogeneous;
a stability of their water-in-oil form after 3 months of storage at a temperature of 45° C.; the observed appearance after this period of storage is still homogeneous;
dynamic viscosity values measured after 3 months of storage at 25° C. by means of a Brookfield LV viscometer at 25° C. and at a speed of 6 rpm of between 27 000 mPa·s (for (F₂)) and 65 000 mPa·s (for (F₁)).

The formulation (F₁) differs from (F₂) by the absence of econazole nitrate within it; (F₁) is in the form of a water-in-oil emulsion while the formulation of (F₂) does not make it possible to obtain a water-in-oil emulsion but rather a water-in-oil emulsion.

When the weight content of oil (Lanol™ 2681) in the formulation is increased to 15% in the formulation (F₁), the emulsion obtained is of the water-in-oil type whereas the emulsion obtained is of the oil-in-water type for the formulation (F2) containing 8% of Lanol™ 2681.

The formulation (F₃), differs from the formulation (F₁) only in the nature of the surfactant, that is to say sorbitan oleate (Montane™ 80) for the formulation (F₃) instead of cetearyl polyglucoside/cetearyl alcohol for the formulation (F₃), does not make it possible to obtain a water-in-oil emulsion after storage at 25° C. and at 45° C. for three months.
The formulation (F4) contains only one water-in-oil surfactant, PEG 30 dipolyhydroxystearate (sold under the trade name SimalineTM WO) which is a water-in-oil surfactant, and does not make it possible to obtain a water-in-oil emulsion which is stable after storage at 45° C. for three months because a phase separation and a heterogeneous appearance are observed from the end of the first month of storage.

1. A pharmaceutical composition (El) suitable for topical use which is in the form of a water-in-oil emulsion comprising, for 100% of the weight thereof:
from 0.3% to 5% by weight of at least one local azole antifungal substance;
from 60% to 90% by weight of a gelled aqueous phase (A1);
from 9.7% to 35% by weight of a fatty phase (A2) comprising at least one oil and an emulsifying system comprising a combination of at least one emulsifying surfactant (S1) chosen from the elements of the group consisting of compositions of alkyl polyglycosides, and compositions of alkyl polyglycosides and of fatty alcohols, and of at least one emulsifying surfactant (S2) chosen from the elements of the group consisting of polyglycerol esters, alkoxylated polyglycerol esters, polyglycol polyhydroxystearates, polyglycerol polyhydroxystearates, and alkoxylated polyglycerol polyhydroxystearates.

2. The pharmaceutical composition of claim 1, wherein the gelled aqueous phase comprises, for 100% of the weight thereof:
from 0.5% to 10% by weight of a crosslinked anionic polyelectrolyte (AP),
from 90% to 99.5% by weight of water.

3. The pharmaceutical composition of claim 2, wherein the crosslinked anionic polyelectrolyte (AP) comprises a proportion of greater than or equal to 25 mol % of monomer units derived from 2-methyl-2-[(1-oxo-2-propenyl)amino]-1-propanesulfonic acid in free acid or partially or totally salified form.

4. The pharmaceutical composition of claim 1, wherein the local azole antifungal substance is chosen from the elements, which are in neutral form or in the form of salts, of the group consisting of (RS)-1-[phenyl(4-phenylphenyl)methyl]-1H-imidazole or bifonazole, in the form of the R enantiomer of formula (Ia₁) (CAS number=91487-85-3) or of the S enantiomer of formula (Ia₂) (CAS number=91487-86-4): 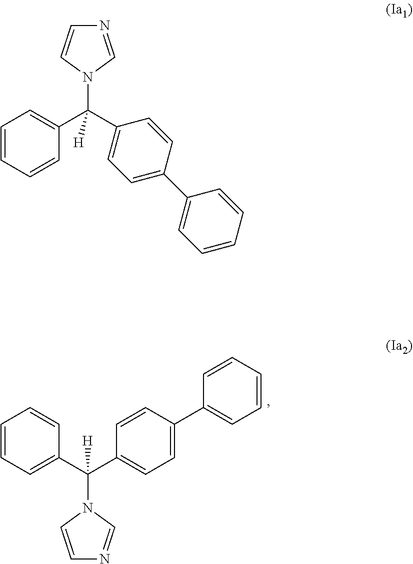 [SMILES: c1ccc(-c2ccc([C@@H](c3ccccc3)n3ccnc3)cc2)cc1 ; c1ccc(-c2ccc([C@H](c3ccccc3)n3ccnc3)cc2)cc1]
(R,S)-1-{2-[(4-chlorophenyl)methoxy]-2-(2,4-dichlorophenyl)ethyl}-1H-imidazole (CAS number=27220-47-9) or econazole, in the form of the R enantiomer of formula (Ib₁) or of the S enantiomer of formula (Ib2): 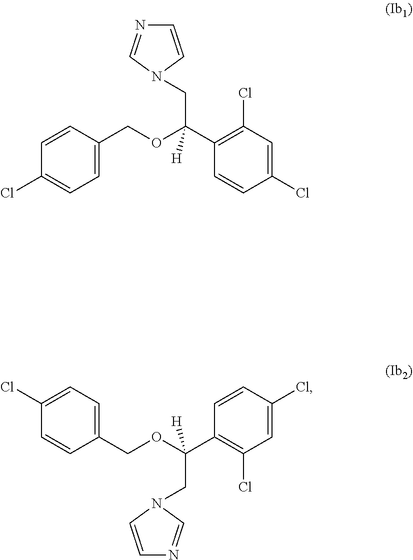 [SMILES: Clc1ccc(CO[C@@H](Cn2ccnc2)c2ccc(Cl)cc2Cl)cc1 ; Clc1ccc(CO[C@H](Cn2ccnc2)c2ccc(Cl)cc2Cl)cc1]
(R,S)-1-[2-(2,4-dichlorophenyl)-2-{[4-(phenylsulfanyl)phenyl]methoxy}ethyl]-1H-imidazole or fenticonazole (CAS number=72479-26-6) of formula (Ic): 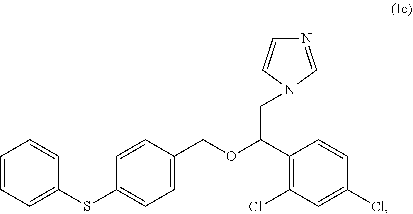 [SMILES: Clc1ccc(C(Cn2ccnc2)OCc2ccc(Sc3ccccc3)cc2)c(Cl)c1]
(R,S)-1-{2-[(2,6-dichlorobenzyl)oxy]-2-(2,4-dichlorophenyl)ethyl}-1H-imidazole or isoconazole (CAS number=27523-40-6) in the form of the R enantiomer of formula (Id₁) or in the form of the S enantiomer of formula (Id2): 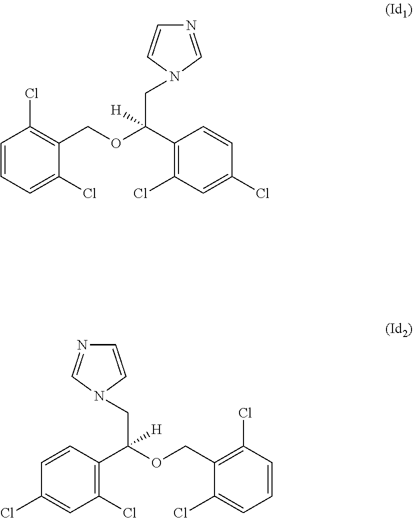 [SMILES: Clc1ccc([C@H](Cn2ccnc2)OCc2c(Cl)cccc2Cl)c(Cl)c1 ; Clc1ccc([C@@H](Cn2ccnc2)OCc2c(Cl)cccc2Cl)c(Cl)c1]
1-[4-(4-{[(2R,4S)-2-(2,4-dichlorophenyl)-2-(1H-imidazol-1-ylmethyl)-1,3-dioxolan-4-yl]methoxy}phenyl)piperazin-1-yl]ethan-1-one or ketoconazole (CAS number=65277-42-1) in the form of the R enantiomer of formula (Ie₁) or in the form of the S enantiomer of formula (Ie₂): 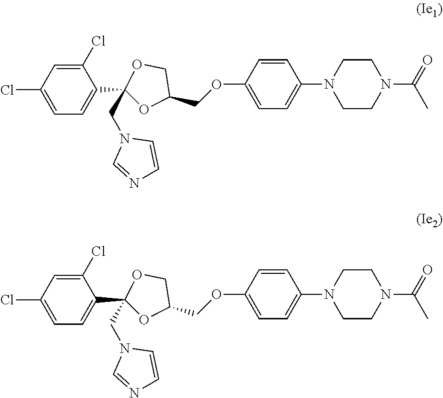 [SMILES: CC(=O)N1CCN(c2ccc(OC[C@@H]3CO[C@@](Cn4ccnc4)(c4ccc(Cl)cc4Cl)O3)cc2)CC1 ; CC(=O)N1CCN(c2ccc(OC[C@H]3CO[C@](Cn4ccnc4)(c4ccc(Cl)cc4Cl)O3)cc2)CC1]
(R,S)-1-{2-[(2,4-dichlorobenzyl)oxy]-2-(2,4-dichlorophenyl)ethyl}-1H-imidazole or miconazole (CAS number=22916-47-8) in the form of the R enantiomer of formula (If₁) or in the form of the S enantiomer of formula (If₂): 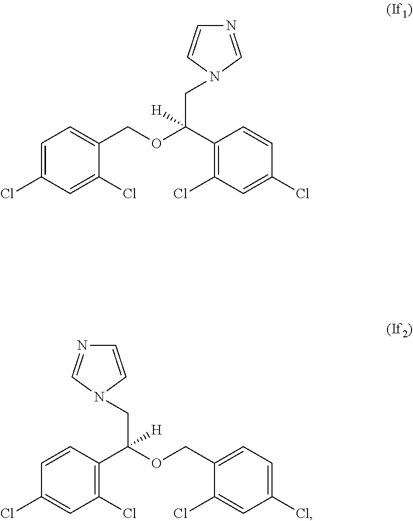 [SMILES: Clc1ccc(CO[C@@H](Cn2ccnc2)c2ccc(Cl)cc2Cl)c(Cl)c1 ; Clc1ccc(CO[C@H](Cn2ccnc2)c2ccc(Cl)cc2Cl)c(Cl)c1]
1-[(Z)-2-[2-(4-chlorophenoxy)ethoxy]-2-(2,4-dichlorophenyl)-1-methylvinyl]-1H-imidazole (CAS number=74512-12-2) or omoconazole of formula (Ig): 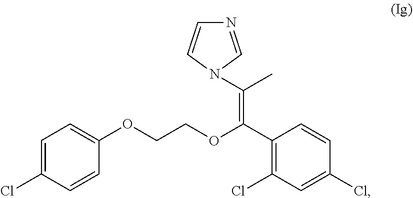 [SMILES: C/C(=C(/OCCOc1ccc(Cl)cc1)c1ccc(Cl)cc1Cl)n1ccnc1]
(1Z)-N-[(2,4-dichlorobenzyl)oxy]-1-(2,4-dichlorophenyl)-2-(1H-imidazol-1-yl)ethanimine or oxiconazole (CAS number=64211-45-6) of formula (Ih): 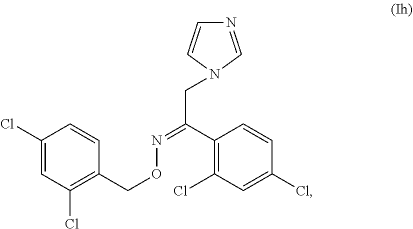 [SMILES: Clc1ccc(CO/N=C(/Cn2ccnc2)c2ccc(Cl)cc2Cl)c(Cl)c1]
(R,S)-1-{2-[(7-chloro-1-benzothiophen-3-yl)methoxy]-2-(2,4-dichlorophenyl)ethyl}-1H-imidazole or sertaconazole (CAS number=99592-32-2) of formula (Ii): 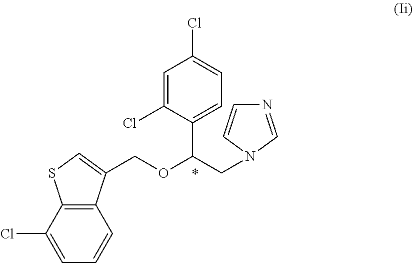 [SMILES: Clc1ccc(C(Cn2ccnc2)OCc2csc3c(Cl)cccc23)c(Cl)c1]
(R,S)-1-{2-[(2-chlorothiophen-3-yl)methoxy]-2-(2,4-dichlorophenyl)ethyl}-1H-imidazole or tioconazole (CAS number=65899-73-2) in the form of the R enantiomer of formula (Ij₁) or in the form of the S enantiomer of formula (Ij₂): 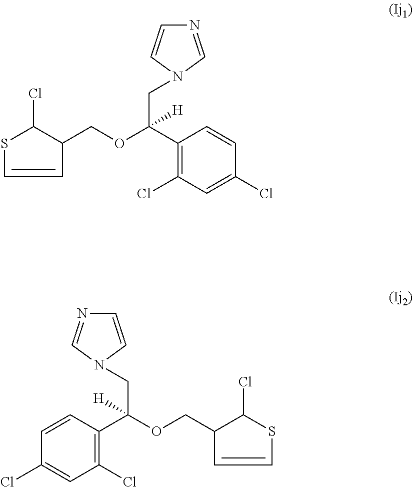 [SMILES: Clc1ccc([C@H](Cn2ccnc2)OCC2C=CSC2Cl)c(Cl)c1 ; Clc1ccc([C@@H](Cn2ccnc2)OCC2C=CSC2Cl)c(Cl)c1]
1-[(2-chlorophenyl)(diphenyl)methyl]-1H-imidazole or clotrimazole (CAS number=23593-75-1) of formula (Ik).

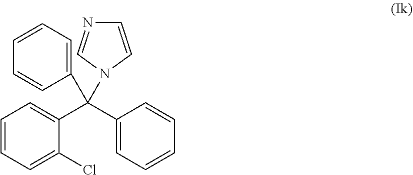 [SMILES: Clc1ccccc1C(c1ccccc1)(c1ccccc1)n1ccnc1]

5. The pharmaceutical composition of claim 1, wherein the local azole antifungal substance is chosen from the elements of the group consisting of the hydrochloride, phosphate, nitrate, propionate and citrate salts of the compounds of formulae (Ia₁), (Ia₂), (Ib₁), (Ib₂), (Ic), (Id₁), (Id₂), (Ie₁), (Ie₂), (If₁), (If₂), (Ig), (Ih), (Ii), (Ij₁), (Ij₂) and (Ik).

6. The pharmaceutical composition of claim 1, wherein the emulsifying surfactant (S₁) consists of at least one alkyl polyglycoside composition (C₁) represented by formula (VIII):
R₁-O-(G)x-H (VIII)
wherein x represents a decimal number between 1.05 and 2.5, G represents the glucosyl or α,β-D-glucopyranosyl radical, obtained from the removal of the hemiacetal hydroxyl group from α,β-D-glucopyranose, and R₁ represents a radical chosen from the elements of the group consisting of the radicals n-dodecyl, n-tetradecyl, n-hexadecyl, n-octadecyl, n-eicosyl and n-behenyl, said composition (C₁) consisting of a mixture of compounds represented by formulae (VIII₁), (VIII₂), (VIII₃), (VIII₄) and (VIII₅):
R₁-O-(G)₁-H (VIII₁)
R₁-O-(G)₂-H (VIII₂)
R₁-O-(G)₃-H (VIII₃)
R₁-O-(G)₄-H (VIII₄)
R₁-O-(G)₅-H (VIII₅),
in the respective molar proportions a₁, a₂, a₃, a₄ and a₅, such that:
the sum a₁+a₂+a₃+a₄+a₅ as is equal to 1, and that
the sum a₁+2a₂+3a₃+4a₄+5a₅ is equal to x.

7. The pharmaceutical composition of claim 1, wherein the emulsifying surfactant (S₁) consists of at least one composition (C₂) comprising, for 100% of the weight thereof:
from 10% to 50% by weight of at least one alkylpolyglycoside composition (C₁) represented by formula (VIII):
R₁-O-(G)x-H (VIII),
wherein x represents a decimal number between 1.05 and 2.5, G represents the glucosyl or α,β-D-glucopyranosyl radical, obtained from the removal of the hemiacetal hydroxyl group from α,β-D-glucopyranose, and R₁ represents a radical chosen from the elements of the group consisting of the radicals n-dodecyl, n-tetradecyl, n-hexadecyl, n-octadecyl, n-eicosyl and n-behenyl, said composition consisting of a mixture of compounds represented by formulae (VIII₁), (VIII₂), (VIII₃), (VIII₄) and (VIII₅):
R1-O-(G)1-H (VIII1)
R1-O-(G)2-H (VIII2)
R1-O-(G)3-H (VIII3)
R1-O-(G)4-H (VIII4)
R1-O-(G)5-H (VIII5)
in the respective molar proportions a₁, a₂, a₃, a₄ and a₅, such that:
the sum a₁+a₂+a₃+a₄+a₅ as is equal to 1, and that
the sum a₁+2a₂+3a₃+4a₄+5a₅ is equal to x; and
from 90% to 50% by weight of at least one fatty alcohol of formula (IX):
R″₁—OH (IX),
wherein R″₁ represents a radical chosen from the elements of the group consisting of the radicals n-dodecyl, n-tetradecyl, n-hexadecyl, n-octadecyl, n-eicosyl and n-behenyl, where R′₁ may be identical to or different from R₁.

8. The pharmaceutical composition of claim 1, wherein the emulsifying surfactant (S₂) consists of at least one polyglycol polyhydroxystearate represented by the formula (XII): 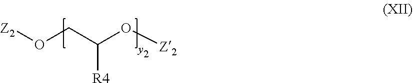
wherein y2 represents an integer greater than or equal to 2 and less than or equal to 50, R₄ represents a hydrogen atom, a methyl radical or an ethyl radical, and Z₂ represents a radical of formula (XIII): 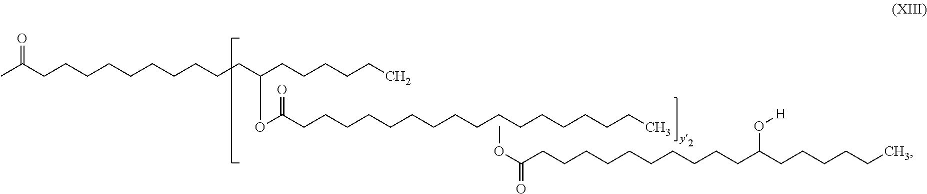
wherein y′₂ represents an integer greater than or equal to 0 and less than or equal to 10, more particularly greater than or equal to 1 and less than or equal to 10, and Z′₂ represents a radical of formula (XIII) as defined above, where Z′₂ may be identical to or different from Z₂, or a hydrogen atom.

9. The pharmaceutical composition of claim 1, wherein the weight ratio between the emulsifying surfactant (S₁) and the emulsifying surfactant (S₂) is greater than or equal to ¼ and less than or equal to 1.

10. The pharmaceutical composition of claim 1, wherein said composition is intended to be used for therapy in a human or animal mammal.

11. A method for treating mycoses of the skin, the scalp, the mouth and/or the gynecological system in human or animal mammals, comprising administering a therapeutically effective dose of the composition of claim 1 to a human or animal mammal in need thereof.

12. The pharmaceutical composition of claim 1, further comprising at least one or more auxiliary compounds chosen from foaming and/or detergent surfactants, thickening and/or gelling surfactants, thickening and/or gelling agents, stabilizers, film-forming compounds, solvents and cosolvents, hydrotropic agents, plasticizing agents, opacifying agents, pearlescent agents, superfatting agents, sequestering agents, chelating agents, antioxidants, fragrances, essential oils, preservatives, conditioning agents, deodorants, and mineral fillers or pigments.

13. The pharmaceutical composition of claim 8, wherein y′₂ represents an integer greater than or equal to 1 and less than or equal to 10.

14. The pharmaceutical composition of claim 2, wherein the local azole antifungal substance is chosen from the elements, which are in neutral form or in the form of salts, of the group consisting of (RS)-1-[phenyl(4-phenylphenyl)methyl]-1H-imidazole or bifonazole, in the form of the R enantiomer of formula (Ia₁) (CAS number=91487-85-3) or of the S enantiomer of formula (Ia₂) (CAS number=91487-86-4): 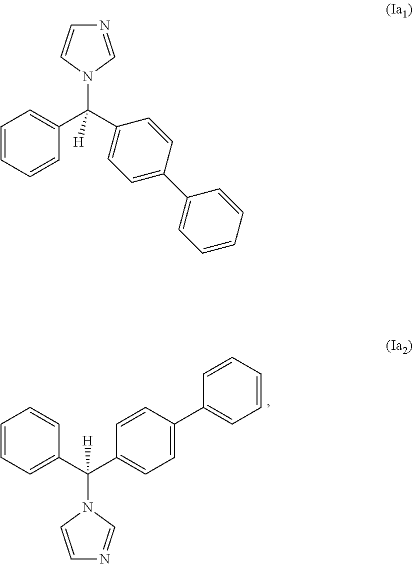 [SMILES: c1ccc(-c2ccc([C@@H](c3ccccc3)n3ccnc3)cc2)cc1 ; c1ccc(-c2ccc([C@H](c3ccccc3)n3ccnc3)cc2)cc1]
(R,S)-1-{2-[(4-chlorophenyl)methoxy]-2-(2,4-dichlorophenyl)ethyl}-1H-imidazole (CAS number=27220-47-9) or econazole, in the form of the R enantiomer of formula (Ib₁) or of the S enantiomer of formula (Ib₂): 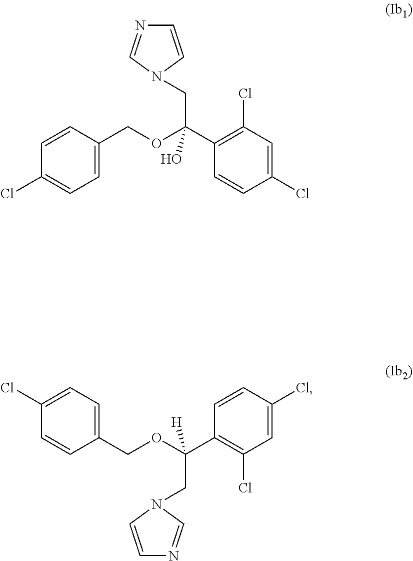 [SMILES: O[C@@](Cn1ccnc1)(OCc1ccc(Cl)cc1)c1ccc(Cl)cc1Cl ; Clc1ccc(CO[C@H](Cn2ccnc2)c2ccc(Cl)cc2Cl)cc1]
(R,S)-1-[2-(2,4-dichlorophenyl)-2-{[4-(phenylsulfanyl)phenyl]methoxy}ethyl]-1H-imidazole or fenticonazole (CAS number=72479-26-6) of formula (Ic): 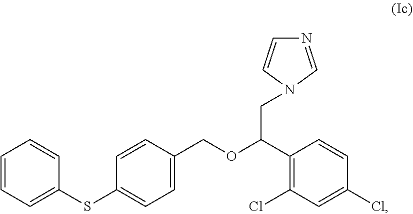 [SMILES: Clc1ccc(C(Cn2ccnc2)OCc2ccc(Sc3ccccc3)cc2)c(Cl)c1]
(R,S)-1-{2-[(2,6-dichlorobenzyl)oxy]-2-(2,4-dichlorophenyl)ethyl}-1H-imidazole or isoconazole (CAS number=27523-40-6) in the form of the R enantiomer of formula (Id₁) or in the form of the S enantiomer of formula (Id₂): 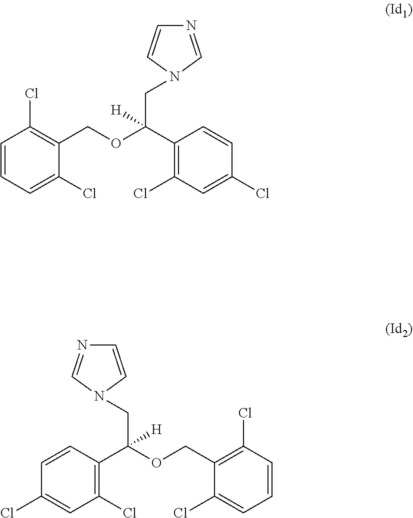 [SMILES: Clc1ccc([C@H](Cn2ccnc2)OCc2c(Cl)cccc2Cl)c(Cl)c1 ; Clc1ccc([C@@H](Cn2ccnc2)OCc2c(Cl)cccc2Cl)c(Cl)c1]
1-[4-(4-{[(2R,4S)-2-(2,4-dichlorophenyl)-2-(1H-imidazol-1-ylmethyl)-1,3 -dioxolan-4-yl]methoxy}phenyl)piperazin-1-yl]ethan-1- one or ketoconazole (CAS number=65277-42-1) in the form of the R enantiomer of formula (Ie₁) or in the form of the S enantiomer of formula (Ie₂): 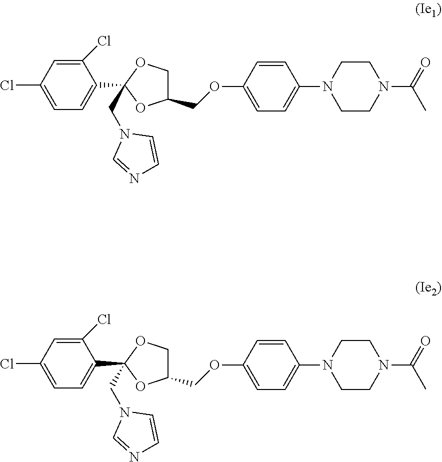 [SMILES: CC(=O)N1CCN(c2ccc(OC[C@@H]3CO[C@@](Cn4ccnc4)(c4ccc(Cl)cc4Cl)O3)cc2)CC1 ; CC(=O)N1CCN(c2ccc(OC[C@H]3CO[C@](Cn4ccnc4)(c4ccc(Cl)cc4Cl)O3)cc2)CC1]
(R,S)-1-{2-[(2,4-dichlorobenzyl)oxy]-2-(2,4-dichlorophenyl)ethyl}-1H-imidazole or miconazole (CAS number=22916-47-8) in the form of the R enantiomer of formula WO or in the form of the S enantiomer of formula (If₂): 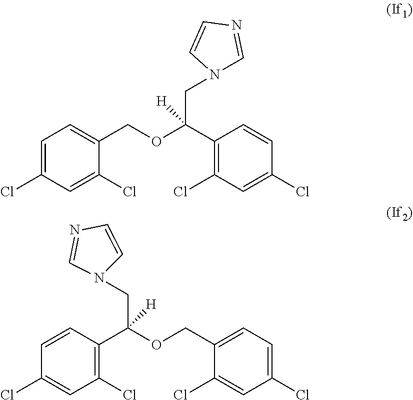
1-[(Z)-2-[2-(4-chlorophenoxy)ethoxy]-2-(2,4-dichlorophenyl)-1-methylvinyl]-1H-imidazole (CAS number=74512-12-2) or omoconazole of formula (Ig): 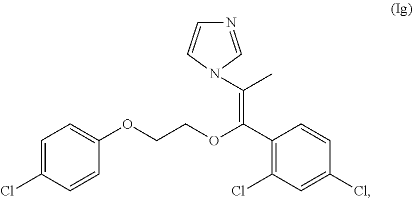 [SMILES: C/C(=C(/OCCOc1ccc(Cl)cc1)c1ccc(Cl)cc1Cl)n1ccnc1]
(1Z)-N-[(2,4-dichlorobenzyl)oxy]-1-(2,4-dichlorophenyl)-2-(1H-imidazol-1-yl)ethanimine or oxiconazole (CAS number=64211-45-6) of formula (Ih): 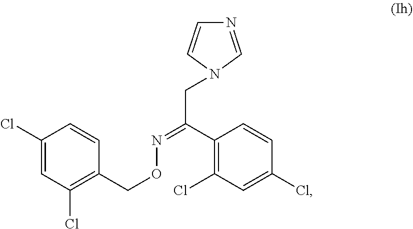 [SMILES: Clc1ccc(CO/N=C(/Cn2ccnc2)c2ccc(Cl)cc2Cl)c(Cl)c1]
(R,S)-1-{2-[(7-chloro-1-benzothiophen-3-yl)methoxy]-2-(2,4-dichlorophenyl)ethyl}-1H-imidazole or sertaconazole (CAS number=99592-32-2) of formula (Ii): 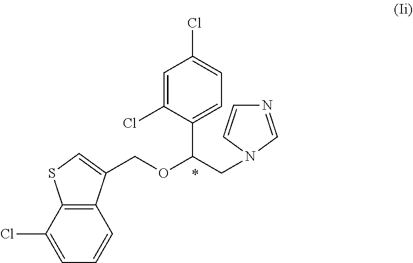 [SMILES: Clc1ccc(C(Cn2ccnc2)OCc2csc3c(Cl)cccc23)c(Cl)c1]
(R,S)-1-{2-[(2-chlorothiophen-3-yl)methoxy]-2-(2,4-dichlorophenyl)ethyl}-1H-imidazole or tioconazole (CAS number=65899-73-2) in the form of the R enantiomer of formula (Ij₁) or in the form of the S enantiomer of formula (Ij₂): 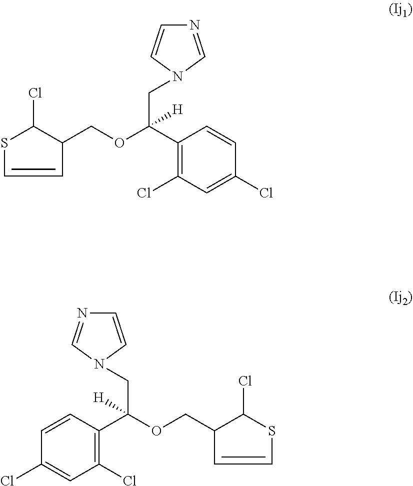 [SMILES: Clc1ccc([C@H](Cn2ccnc2)OCC2C=CSC2Cl)c(Cl)c1 ; Clc1ccc([C@@H](Cn2ccnc2)OCC2C=CSC2Cl)c(Cl)c1]
1-[(2-chlorophenyl)(diphenyl)methyl]-1H-imidazole or clotrimazole (CAS number=23593-75-1) of formula (Ik).

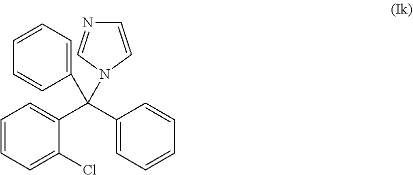 [SMILES: Clc1ccccc1C(c1ccccc1)(c1ccccc1)n1ccnc1]

15. The pharmaceutical composition of claim 3, wherein the local azole antifungal substance is chosen from the elements, which are in neutral form or in the form of salts, of the group consisting of (RS)-1-[phenyl(4-phenylphenyl)methyl]-1H-imidazole or bifonazole, in the form of the R enantiomer of formula (Ia₁) (CAS number=91487-85-3) or of the S enantiomer of formula (Ia₂) (CAS numbe=91487-86-4): 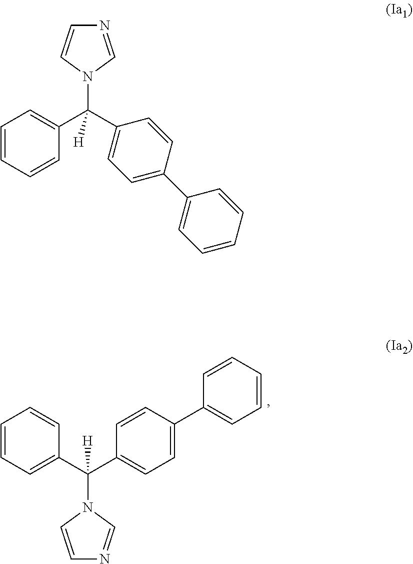 [SMILES: c1ccc(-c2ccc([C@@H](c3ccccc3)n3ccnc3)cc2)cc1 ; c1ccc(-c2ccc([C@H](c3ccccc3)n3ccnc3)cc2)cc1]
(R,S)-1-{2-[(4-chlorophenyl)methoxy]-2-(2,4-dichlorophenyl)ethy}-1H-imidazole (CAS number=27220-47-9) or econazole, in the form of the R enantiomer of formula (Ib₁) or of the S enantiomer of formula (Ib₂): 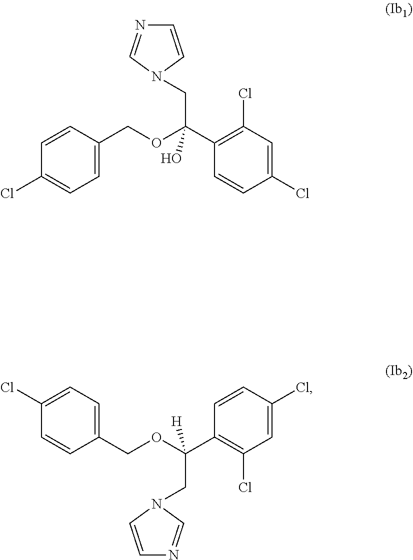 [SMILES: O[C@@](Cn1ccnc1)(OCc1ccc(Cl)cc1)c1ccc(Cl)cc1Cl ; Clc1ccc(CO[C@H](Cn2ccnc2)c2ccc(Cl)cc2Cl)cc1]
(R,S)-1-[2-(2,4-dichlorophenyl)-2-{[4-(phenylsulfanyl)phenyl]methoxy}ethyl]-1H-imidazole or fenticonazole (CAS number=72479-26-6) of formula (Ic): 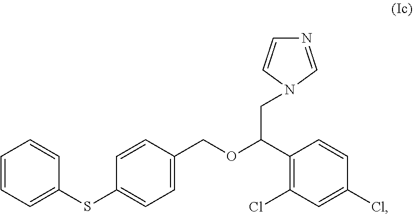 [SMILES: Clc1ccc(C(Cn2ccnc2)OCc2ccc(Sc3ccccc3)cc2)c(Cl)c1]
(R,S)-1-{2-[(2,6-dichlorobenzyl)oxy]-2-(2,4-dichlorophenyl)ethyl}-1H-imidazole or isoconazole (CAS number=27523-40-6) in the form of the R enantiomer of formula (Id₁) or in the form of the S enantiomer of formula (Id₂): 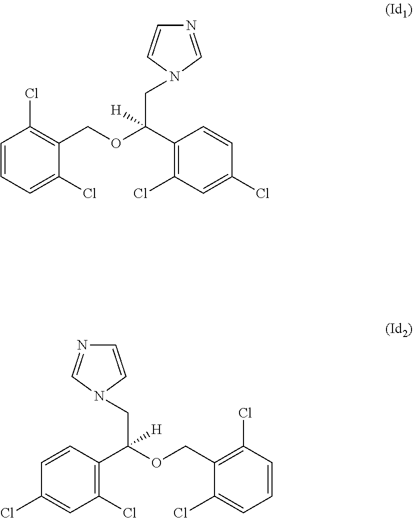 [SMILES: Clc1ccc([C@H](Cn2ccnc2)OCc2c(Cl)cccc2Cl)c(Cl)c1 ; Clc1ccc([C@@H](Cn2ccnc2)OCc2c(Cl)cccc2Cl)c(Cl)c1]
1-[4-(4-{[(2R,4S)-2-(2,4-dichlorophenyl)-2-(1H-imidazol-1-ylmethyl)-1,3-dioxolan-4-yl]methoxy}phenyl)piperazin-1-yl]ethan-1-one or ketoconazole (CAS number=65277-42-1) in the form of the R enantiomer of formula (Iei) or in the form of the S enantiomer of formula (Ie2): 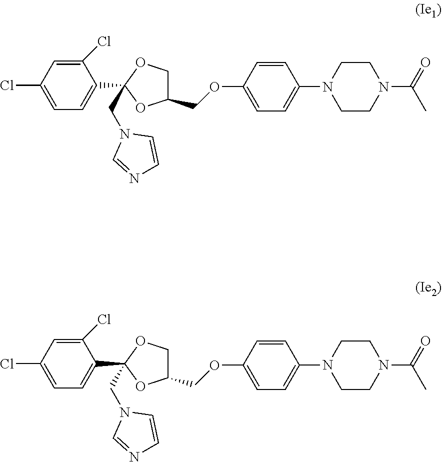 [SMILES: CC(=O)N1CCN(c2ccc(OC[C@@H]3CO[C@@](Cn4ccnc4)(c4ccc(Cl)cc4Cl)O3)cc2)CC1 ; CC(=O)N1CCN(c2ccc(OC[C@H]3CO[C@](Cn4ccnc4)(c4ccc(Cl)cc4Cl)O3)cc2)CC1]
(R,S)-1-{2-[(2,4-dichlorobenzyl)oxy]-2-(2,4-dichlorophenyl)ethyl}-1H-imidazole or miconazole (CAS number=22916-47-8) in the form of the R enantiomer of formula (If₁) or in the form of the S enantiomer of formula (If₂): 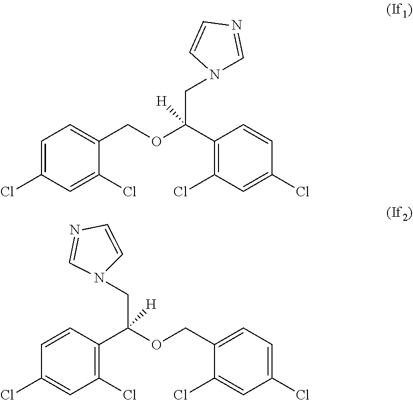
1-[(Z)-2-[2-(4-chlorophenoxy)ethoxy]-2-(2,4-dichlorophenyl)-1-methylvinyl]-1H-imidazole (CAS number=74512-12-2) or omoconazole of formula (Ig): 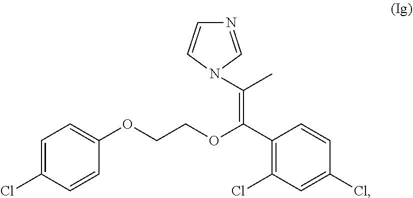 [SMILES: C/C(=C(/OCCOc1ccc(Cl)cc1)c1ccc(Cl)cc1Cl)n1ccnc1]
(1Z)-N-[(2,4-dichlorobenzyl)oxy]-1-(2,4-dichlorophenyl)-2-(1H-imidazol-1-yl)ethanimine or oxiconazole (CAS number=64211-45-6) of formula (Ih): 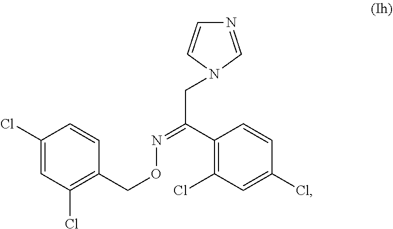 [SMILES: Clc1ccc(CO/N=C(/Cn2ccnc2)c2ccc(Cl)cc2Cl)c(Cl)c1]
(R,S)-1-{2-[(7-chloro-1-benzothiophen-3-yl)methoxy]-2-(2,4-dichlorophenyl)ethyl}-1H-imidazole or sertaconazole (CAS number=99592-32-2) of formula (Ii): 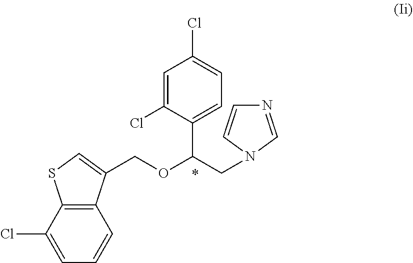 [SMILES: Clc1ccc(C(Cn2ccnc2)OCc2csc3c(Cl)cccc23)c(Cl)c1]
(R,S)-1-{2-[(2-chlorothiophen-3-yl)methoxy]-2-(2,4-dichlorophenyl)ethyl}-1H-imidazole or tioconazole (CAS number=65899-73-2) in the form of the R enantiomer of formula (Ij₁) or in the form of the S enantiomer of formula (Ij₂): 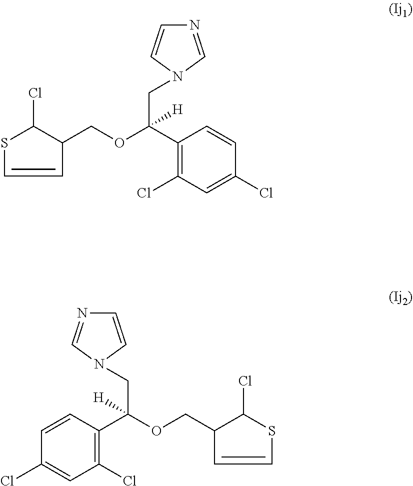 [SMILES: Clc1ccc([C@H](Cn2ccnc2)OCC2C=CSC2Cl)c(Cl)c1 ; Clc1ccc([C@@H](Cn2ccnc2)OCC2C=CSC2Cl)c(Cl)c1]
1-[(2-chlorophenyl)(diphenyl)methyl]-1H-imidazole or clotrimazole (CAS number=23593-75-1) of formula (Ik).

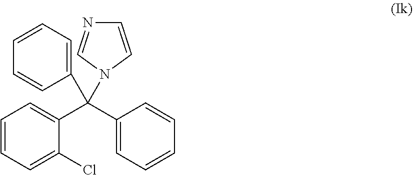 [SMILES: Clc1ccccc1C(c1ccccc1)(c1ccccc1)n1ccnc1]

16. The pharmaceutical composition of claim 2, wherein the local azole antifungal substance is chosen from the elements of the group consisting of the hydrochloride, phosphate, nitrate, propionate and citrate salts of the compounds of formulae (Ia₁), (Ia₂), (Ib₁), (Ib₂), (Ic), (Id₁), (Id₂), (Ie₁), (Ie₂), (If₁), (If₂), (Ig), (Ih), (Ii), (Ij₁), (Ij₂) and (Ik).

17. The pharmaceutical composition of claim 3, wherein the local azole antifungal substance is chosen from the elements of the group consisting of the hydrochloride, phosphate, nitrate, propionate and citrate salts of the compounds of formulae (Ia₁), (Ia₂), (Ib₁), (Ib₂), (Ic), (Id₁), (Id₂), (Ie₁), (Ie₂), (If₁), (If₂), (Ig), (Ih), (Ii), (Ij₁), (Ij₂) and (Ik).

18. The pharmaceutical composition of claim 4, wherein the local azole antifungal substance is chosen from the elements of the group consisting of the hydrochloride, phosphate, nitrate, propionate and citrate salts of the compounds of formulae (Ia₁), (Ia₂), (Ib₁), (Ib₂), (Ic), (Id₁), (Id₂), (Ie₁), (Ie₂), (If₁), (If₂), (Ig), (Ih), (Ii), (Ij₁), (Ij₂) and (Ik).

19. The pharmaceutical composition of claim 2, wherein the emulsifying surfactant (S₁) consists of at least one alkyl polyglycoside composition (C₁) represented by formula (VIII):
R₁-O-(G)x-H (VIII)
wherein x represents a decimal number between 1.05 and 2.5, G represents the glucosyl or α,β-D-glucopyranosyl radical, obtained from the removal of the hemiacetal hydroxyl group from α,β-D-glucopyranose, and R₁ represents a radical chosen from the elements of the group consisting of the radicals n-dodecyl, n-tetradecyl, n-hexadecyl, n-octadecyl, n-eicosyl and n-behenyl, said composition (C₁) consisting of a mixture of compounds represented by formulae (VIII₁), (VIII₂), (VIII₃), (VIII₄) and (VIII₅):
R₁-O-(G)₁-H (VIII₁)
R₁-O-(G)₂-H (VIII₂)
R₁-O-(G)₃-H (VIII₃)
R₁-O-(G)₄-H (VIII₄)
R₁-O-(G)₅-H (VIII₅),
in the respective molar proportions a₁, a₂, a₃, a₄ and a₅, such that:
the sum a₁+a₂+a₃+a₄+a₅ as is equal to 1, and that
the sum a₁+2a₂+3a₃+4a₄+5a₅ is equal to x.

20. The pharmaceutical composition of claim 3, wherein the emulsifying surfactant (S₁) consists of at least one alkyl polyglycoside composition (C₁) represented by formula (VIII):
R₁-O-(G)x-H (VIII)
wherein x represents a decimal number between 1.05 and 2.5, G represents the glucosyl or α,β-D-glucopyranosyl radical, obtained from the removal of the hemiacetal hydroxyl group from α,β-D-glucopyranose, and R₁ represents a radical chosen from the elements of the group consisting of the radicals n-dodecyl, n-tetradecyl, n-hexadecyl, n-octadecyl, n-eicosyl and n-behenyl, said composition (C₁) consisting of a mixture of compounds represented by formulae (VIII₁), (VIII₂), (VIII₃), (VIII₄) and (VIII₅):
R₁-O-(G)₁-H (VIII₁)
R₁-O-(G)₂-H (VIII₂)
R₁-O-(G)₃-H (VIII₃)
R₁-O-(G)₄-H (VIII₄)
R₁-O-(G)₅-H (VIII₅),
in the respective molar proportions a₁, a₂, a₃, a₄ and a₅, such that:
the sum a₁+a₂+a₃+a₄+a₅ is equal to 1, and that
the sum a₁+2a₂+3a₃+4a₄+5a₅ is equal to x.
